(19) United States
(12) Patent Application Publication
(10) Pub. No.: US 2007/0293517 A1
(43) Pub. Date: Dec. 20, 2007

(54) Derivatives Of Chemotherapeutic Agents With A Formaldehyde Releasing Moiety

(75) Inventors: Abraham Nudelman, Rechovot (IL); Ada Rephaeli, Herzlia (IL)

Correspondence Address: Martin D. Moynihan;PRTSI, P.O.Box 16446, Arlington, VA, 22215, US

(73) Assignee: RAMOT AT TEL AVIV UNIVERSITY LTD., TEL-AVIV, ISRAEL (IL)

(21) Appl. No.: 11/597,829

(22) PCT Filed: Jun. 9, 2005
(86) PCT No.: PCT/IL05/00614

§ 371 Date: Nov. 28, 2006

Related U.S. Application Data
(60) Provisional application No. 60/577,921, filed on Jun. 9, 2004.

(51) Int. Cl. A61K 31/513; A61K 31/45; A61K 31/454; A61P 31/00; A61P 31/10; A61P 35/00; C07D 239/553; C07D 473/38; C07D 401/04; A61P 37/00; A61P 31/12; A61P 31/04; A61K 31/522; A61K 31/506

(52) U.S. Cl. 514/263.3; 514/274; 514/323; 544/265; 544/276; 544/313; 546/201

(57) ABSTRACT

Novel conjugates of chemotherapeutic agents, which are designed so as to release formaldehyde or analogs thereof upon cleavage, processes of preparing same, pharmaceutical compositions containing same and methods utilizing same for treating medical conditions such as cancer, immune-mediated diseases, viral infections and diseases, bacterial infections and diseases, fungal infections and diseases, protozal infections and diseases and more, and particularly for treating such conditions which are characterized by drug resistance are provided.

### RELATED APPLICATIONS

This application is a National Phase Application of PCT Application No. PCT/IL2005/000614 having International Filing Date of Jun. 9, 2005, which claims the benefit of U.S. Provisional Patent Application No. 60/577,921, filed on Jun. 9, 2004. The contents of the above Applications are all incorporated herein by reference.

### FIELD AND BACKGROUND OF THE INVENTION

The present invention relates to the field of chemotherapy, and more particularly, to novel derivatives of widely used chemotherapeutic agents which exhibit enhanced therapeutic efficacy, to pharmaceutical compositions containing same and to uses thereof in the treatment of various diseases, including cancer, viral, bacterial and fungal diseases.

Chemotherapy is a collective term that is widely used in the art to describe the use of chemical compounds (also referred to herein and in the art interchangeably as “chemicals” or simply “compounds”) to treat diseases. Chemical compounds that are used in chemotherapy are collectively referred to in the art as chemotherapeutic agents. There are many different types of chemical compounds that qualify as chemotherapeutic agents, and these are classified into several major categories based on the way the compounds affect cell chemistry and on the stage of the cells life cycle that the compounds affect. Common classes of chemotherapeutic agents include, for example, anti-cancer agents, antiviral agents, antimycotic agents and antimicrobial agents.

Chemotherapeutic agents that are typically used in the treatment of cancer and other proliferative disorders are categorized, for example, as alkylating agents, nitrosoureas, antimetabolites, antitumorantibiotics, plant alkaloids, platinum coordination complexes or steroid hormones. Examples of widely used anticancer chemotherapeutic agents (which are also referred to, interchangeably, herein and in the art, as antineoplastic agents) are tegafur, 5-fluorouracil (5-FU), uracil, doxorubicin, 6-mercaptopurine and thalidomide.

The use of chemotherapeutic agents in treating cancer is oftentimes severely limited by the appearance of cells that are resistant to the agents, and/or by the adverse side effects associated therewith. Many of the most prevalent forms of human cancer resist chemotherapeutic intervention. Some tumor populations, especially adrenal, colon, jejunal, kidney and liver carcinomas, appear to have drug-resistant cells at the outset of treatment (Barrows, L. R., “Antineoplastic and Immunoactive Drugs”, Chapter 75, pp 1236-1262, in: Remington: The Science and Practice of Pharmacy, Mack Publishing Co. Easton, Pa., 1995). In other cases, a resistance-conferring genetic change occurs during treatment; the resistant daughter cells then proliferate in the environment of the chemotherapeutic agent. Whatever the cause, resistance often terminates the usefulness of an antineoplastic drug.

Another prevalent problem that is associated with tumor chemotherapy is the appearance of adverse side affects upon the administration of a chemotherapeutic agent. These adverse side effects typically result from the fact that the chemotherapeutic agents, by attacking proliferating cancerous cells attack also normal, non-cancerous proliferating cells.

Anticancer drug resistance may be either intrinsic or acquired. The multidrug resistance (MDR) is a unique phenomenon and is characterized by tumor resistance to various structurally unrelated chemotherapeutic agents. Known mechanisms for MDR include over-expression of a membrane P-glycoprotein 170 and elevated cellular levels of reducing agents, such as glutathione (GSH).

Currently available strategies for overcoming drug resistance include chemical modifications of the treatment, competitive inhibitors of the P-glycoprotein 170, inhibitors of GSH synthesis, and adjuvant therapy with hyperthermia. Development of drug resistance is analogous to a physiological detoxification mechanism and may continue to limit the effectiveness of cancer chemotherapy in the near future (Mansouri et al., SAAS Bull Biochem Biotechnol. 1990 January;3:91-6).

Chemical modification of cancer treatment involves the use of agents or maneuvers that are not cytotoxic in themselves, but modify the host or tumor so as to enhance anticancer therapy and/or selectively protect non-cancer cells from the effects of cytotoxic drugs. Such agents are called chemosensitizers and chemoprotectors, respectively. However, all the presently known agents and methods for reducing drug resistance in cancer therapy have not provided yet a comprehensive and efficient solution to combat anticancer drug resistance.

Immunomodulating agents are also an important and much intriguing group of chemotherapeutic agents. An example of a common immunomodulating chemotherapeutic agent is Azathioprine, which was originally developed for the control of graft rejection in transplant surgery. Since it became available in the early 1960s, azathioprine has also been prescribed for a range of autoimmune and immune-mediated dermatological conditions such as dermatomyositis, systemic lupus erythematosus and pemphigus vulgaris. It is used for treating these conditions either alone or, more commonly, in combination with corticosteroids. The azathioprine (AZA) serves as a prodrug, which upon entering the body is rapidly reduced in the presence of glutathione to 6-mercaptopurine (6-MP) and then metabolized into active metabolites with immune-modifier activity. Azathioprine is also frequently used as monotherapy for atopic eczema (Anstey A V, Wakelin S, Reynolds N J. British Association of Dermatologists Therapy, Guidelines and Audit Subcommittee. Guidelines for prescribing azathioprine in dermatology. Br J Dermatol. 2004 151, 1123-32). Other effective immunomodulating agents are, for example, methotrexate and sulfasalazine. Additional, less effective, immunomodulating agents include the antimalarials, colchicine, cyclosporine, and the retinoids.

Immunomodulating agents usually act by inhibiting the function of T cells, which are an important component of the immune system. However, these agents are slow to act, and a benefit may not be seen for 2 to 4 months after onset of the treatment. Furthermore, immunomodulating agent treatments are oftentimes associated with adverse side effects such as skin rash, loss of appetite, nausea, vomiting, diarrhea, flulike symptoms, chest discomfort, palpitations, and dyspnea. Furthermore, after long term usage more serious side effects may develop, such as bone marrow depression, liver damage, drug-induced pneumonia, pancreatitis and, in rare cases, even leukopenia and elevated transaminases. Thus, treatment with immunomodulating agents requires close monitoring by the physician. While immunomodulating therapy has the potential to suppress joint inflammation and preserve functional capacity, a true disease modification has yet to be shown. The toxicity associated with presently available immunomodulating agents makes careful patient selection and conscientious monitoring essential.

Antiviral chemotherapeutic agents act either by directly inactivating intact viruses, by altering the host immune responses to infection, or by inhibiting viral replication at the cellular level, interrupting one or more steps in the life cycle of the virus. Known antiviral agents are, for example, acyclovir for the treatment of genital Herpes Simplex Virus (HSV), Idoxuridine and trifluridine for the treatment of HSV, gangciclovir for the treatment of Cytomegalovirus (CMV), and stavudine and zidovudine for the treatment of Human Immunodeficiency Virus (HIV).

Antiviral agents have a limited spectrum of activity and, because these agents typically interrupt host cell function, they are toxic to various degrees. A further problem in the use of antiviral agents is the emergence of drug resistant viruses which develops during clinical use and further limits the effectiveness of these agents. For example, the four nucleoside analog chemotherapeutic agents now available for the therapy of HIV do not prevent the ultimate worsening of disease.

Current strategies for coping with the emergence of viral drug-resistant strains include combination drug therapies, for example using chemotherapeutic agents aimed against different viral proteins or chemotherapeutic agents aimed at more than one site on the same protein. Combination therapies may offer additional advantages over monodrug therapy, such as improved antiviral activity and use of lower, less toxic doses. Combinations of various antiviral agents have been extensively studied for HIV and have been effective in the treatment of other diseases caused by infectious agents (e.g., Mycobacterium tuberculosis and Pseudomonas aeruginosa) (K. L. Goldenthal et al., Control of Viral Infections and Diseases, in: http://gsbs.utmb.edu/microbook/ch051.htm). However, combination therapies can also become ineffectual since the virus can develop a complete resistance to the chemotherapeutic agents in a relatively short period of time (see, for example, Birch (1998) AIDS 12:680-681; Roberts (1998) AIDS 12:453-460).

Antimicrobial agents, which are also referred to interchangeably, herein and in the art as “antibacterial agents” or “antibiotics”, act by interfering with (i) cell wall synthesis; (ii) plasma membrane integrity; (iii) nucleic acid synthesis; (iv) ribosomal function; and (v) folate synthesis. Antimicrobial agents that inhibit cell wall synthesis include, for example, β-lactams, such as penicillins and cephalosporins, which inhibit peptidoglycan polymerization, and vancomycin, which combines with cell wall substrates. Antimicrobial agents that interfere with plasma membrane integrity include, for example, polymyxins, which disrupt the plasma membrane, causing leakage, and polyenes (amphotericin) and imidazoles, which attack the plasma membrane sterols of fungi. Antimicrobial agents that interfere with nucleic acid synthesis include, for example, quinolones, which bind to a bacterial complex of DNA and DNA gyrase, blocking DNA replication, nitroimidazoles, which damage DNA, rifampin, which blocks RNA synthesis by binding to DNA directed RNA polymerase. Antimicrobial agents that interfere with the ribosomal function include, for example, aminoglycosides, tetracycline, chloramphenicol, erythromycin, and clindamycin all interfere with ribosome function. Antimicrobial agents that block the folate required for DNA replication include, for example, sulfonamides and trimethoprim.

One of the most prevalent limitations associated with the presently available antibiotics is the evolvement of resistance thereto. Resistance factors can be encoded on plasmids or on the chromosome. Resistance may involve decreased entry of the chemotherapeutic agent into the microorganism's cells, changes in the receptor (target) of the chemotherapeutic agent, or metabolic inactivation of the chemotherapeutic agent. Other limitations include the toxicity of antibiotics and alterations of the normal intestinal flora which may result in diarrhea or in superinfection with opportunistic pathogens.

The rapid spread of antibiotic resistance in pathogenic bacteria has prompted a continuing search for new agents that exhibit antibacterial activity. Indeed, microbiologists today warn of a “medical disaster” which could lead back to the era before penicillin, when even seemingly small infections were potentially lethal. Thus, research into the design of new antibiotics is of high priority. One way to delay the emergence of antibiotic-resistance is to develop new synthetic materials that can selectively inhibit bacterial enzymes, via novel mechanisms of action. However, this approach is both time-consuming and financially prohibitive, yet remains indispensable if an acceptable level of care is to be provided in the immediate future. On the other hand, it may be less costly in time and money to employ strategies to circumvent existing bacterial resistance mechanisms and thereby to restore usefulness to antibacterials that have become compromised by resistance. The remarkable advances in recent years in elucidating the mechanisms of resistance to various clinical antibiotics on the molecular level provide complementary tools to this approach via structure-based and mechanism-based design.

Antimycotic (anti fungal) agents can be divided into several categories: (i) allylamines and other non-azole ergosterol biosynthesis inhibitors, which basically lead to reduced ergosterol biosynthesis and are similar to the azole antifungal agents, described below. One example is terbinafine, which acts by inhibiting squalene epoxidase; (ii) antimetabolites such as flucytosine, which is a DNA substrate analog that leads to incorrect DNA synthesis; (iii) azoles which inhibit the synthesis of ergosterol by blocking the action of 14-alpha-demethylase. An example of an azole anti fungal agent is Fluconazole; (iv) glucan synthesis inhibitors, which inhibit the synthesis of glucan, a key component of the fungal cell wall. Inhibition of this enzyme produces significant antifungal effects. An example of Glucan Synthesis inhibitors is Caspofungin; (v) polyenesa, which are potent agents that act by binding to the fungal cell membrane and causing the fungus to leak electrolytes. One such agent is amphotericin B; and (vi) miscellaneous systemic agents, such as griseofulvin, which act by disrupting the mitotic spindle.

One of the longest known antifungal agents is 5-fluorocytosine (flucytosine; 5-FC), a fluorinated analogue of cytosine. In addition to its activity against Candida spp. and Cryptococcus neoformans, 5-FC is active against fungi causing chromoblastomycosis. 5-FC is now used increasingly in combination with a number of azole antifungal agents, such as ketoconazole, fluconazole and itraconazole. It also plays an important role in the treatment of certain tumors, especially colorectal carcinoma (see http://jac.oupjournals.org/cgilcontent/full/46/2/171).

The occurrence of resistance to 5-FC has been widely described and has led to preclusion of 5-FC as a single agent. Two basic yet distinct mechanisms have been proposed for the resistance evolvement: (i) certain mutations can result in a deficiency in the enzymes necessary for cellular transport and uptake of 5-FC or for its metabolism (i.e. cytosine permease, uridine monophosphate pyrophosphorylase or cytosine deaminase); (ii) resistance may result from increased synthesis of pyrimidines, which compete with the fluorinated antimetabolites of 5-FC and thus diminish its antimycotic activity. It has been shown that defective uridine monophosphate pyrophosphorylase is the most frequently occurring type of acquired 5-FC resistance in fungal cells.

Antiprotozal agents are comprised of several families of compounds, including (i) imidazoles (e.g. metronidazole) and tinidazole, often used against entamoeba, giardia and trichomonas, and interfere with several protozoa cell metabolic pathways; (ii) pyrimethamine, known for the treatment of malaria and toxoplasma, and acts by inhibiting folic acid reduction; (iii) pentamidine, known for the treatment of pneumocystis, trypanosoma rhodesiense and gambiense, which acts by inhibiting aerobic glycolysis; and (iv) chloroquine and quinine, which are known antimalaria agents, and act by inhibiting nucleic acid synthesis.

Treatment of helminth infections can often be complicated due to the complexity of the host-parasite relationship. Most available anti-helminthic chemotherapeutic agents act by interference either with energy metabolism, neuromuscular coordination, microtubular function or with cellular permeability of the parasite. The most commonly used modern anti-helminthics include benzimidazoles, nicotinic agonists, praziquantel, triclabendazole and the macrocyclic lactones. These chemotherapeutic agents interfere with target sites that are either unique to the parasite or differ in their structural features from those of the homologous counterpart present in the vertebrate host. The benzimidazoles exert their effect by binding selectively and with high affinity to the beta-subunit of helminth microtubule protein. The target site of the nicotinic agonists (e.g. levamisole, tetrahydropyrimidines) is a pharmacologically distinct nicotinic acetylcholine receptor channel in nematodes. The macrocyclic lactones (e.g. ivermectin, moxidectin) act as agonists of a family of invertebrate-specific inhibitory chloride channels that are activated by glutamic acid. The primary mode of action of other important antihelminthics (e.g. praziquantel, triclabendazole) is unknown. Antihelminthic resistance is wide-spread and poses a serious threat to effective control of helminth infections, especially in the veterinary area. The biochemical and genetic mechanisms underlying antihelminthic resistance are not well understood, but appear to be complex and vary among different helminth species and even isolates. The major mechanisms helminths use to acquire drug resistance appear to be through receptor loss or decrease of the target site affinity for the drug (Kohler P., The biochemical basis of Antihelminthic action and resistance; Int J Parasitol. 2001 April; 31(4):336-45).

Resistance to antiprotozal and antihelminthic agents has also been widely reported in Upcroft P., Multiple drug resistance in the pathogenic protozoa; Acta Trop. 1994 March; 56(2-3):195-212.

The various studies on the possible mechanisms of chemotherapeutic agent resistance can be summarized to involve one of the following mechanisms:

Drug resistance in cases where the chemotherapeutic agent target remains unchanged typically involves loss of chemotherapeutic agent accumulation mechanism (decreased import) and/or increased chemotherapeutic agent elimination (increases export). This mechanism is found, for example, in multi drug resistance (MDR) cancer cells.

Drug resistance associated with metabolism of the chemotherapeutic agent typically involves conversion of an active chemotherapeutic agent to an inactive form (as in the case of penicilline and penicillinase) and/or non-conversion of prodrugs to the active form (as in the case of certain purine analogues).

Drug resistance associated with alterations in the chemotherapeutic agent target typically involves elimination of the target (e.g. induction of alternative pathway); alteration of the affinity between the target and the chemotherapeutic agent; overproduction of the target (e.g. gene amplification); and accumulation of metabolite antagonistic to the chemotherapeutic agent.

Thus, it is evident that development of resistance is of major consequences in every chemotherapeutical field including, but not limited to, the fields of anticancer, antiviral, antimicrobial, antibacterial, antibiotic, antimycotic, antifungal, antiprotozal and antihelminthic chemotherapies, and attempts are continuously made in order to develop new strategies to combat this problem.

In addition, there is a continuing interest in the modification of undesirable properties of common chemotherapeutic agents, such as low oral chemotherapeutic agent absorption, lack of site specificity, chemical instability, toxicity, and poor patient acceptance (bad taste, odor, pain at injection site, etc.), which may become pharmacological, pharmaceutical, or pharmacokinetic barriers in clinical chemotherapeutic agent application.

The term “prodrug” or “proagent” was first introduced by Albert to signify pharmacologically inactive chemical derivatives that could be used to alter the physicochemical properties of drugs, or chemotherapeutic agents, in a temporary manner, to increase their usefulness and/or to decrease associated toxicity (Albert A. Chemical aspects of selective toxicity. Nature. 1958; 182:421-423). Since then such compounds have also been called “latentiated drugs,” “bioreversible derivatives,” and “congeners,” (Harper N J. Drug latentiation. Prog Drug Res. 1962; 4:221-294; Roche E B. Design of Biopharmaceutical Properties through Prodrugs and Analogs. Washington, D.C.: American Pharmaceutical Association; 1977; Sinkula A A, Yalkowsky S H. Rationale for design of biologically reversible drug derivatives: prodrugs. J Pharm Sci. 1975; 64:181-210). Usually, the use of the term implies a covalent link between a chemotherapeutic agent and a chemical moiety, though in some cases it is used to characterize some forms of salts of the active drug molecule. Although there is no strict universal definition for a prodrug itself, generally, prodrugs can be defined as pharmacologically inert chemical derivatives that can be converted in vivo to the active drug molecules, enzymatically or nonenzymatically, so as to exert a therapeutic effect. Ideally, the prodrug is converted to the original drug once it approaches the targeted organ or tissue, and the released derivatizing group is rapidly eliminated thereafter (see detailed review: Han H. K. and Amidon G. L., Targeted Prodrug Design to Optimize Drug Delivery in AAPS Pharm Sci. 2000; 2 (1): article 6).

The prodrug approach can be useful in the optimization of the clinical application of a chemotherapeutic agent. This approach gained attention as a technique for improving drug therapy in the early 1970s and since then numerous prodrugs have been designed and developed (see for example Stella V. Pro-drugs: an overview and definition. In: Higuchi T, Stella V, eds. Prodrugs As Novel Drug Delivery Systems. ACS Symposium Series. Washington, D.C.: American Chemical Society; 1975:1-115). Following are several examples of prodrug modifications of anticancer, antiviral, antibacterial and antifungal chemotherapeutic agents:

U.S. Pat. No. 5,856,481 (to Nestor et al.) discloses the L-monovaline ester derived from the free hydroxyl groups of the antiviral agent 2-(2-amino-1,6-dihydro-6-oxo-purin-9-yl)methoxy-1,3-propanediol (gangciclovir) and its pharmaceutically acceptable salts. GB Patent No. 2,122,618 discloses derivatives of 9-(2-hydroxyethoxymethyl)guanine which were useful for the treatment of viral infections. GB 2,104,070 discloses similar antiviral compounds. In both British patents the esterification occurs on the hydroxyl groups on the antiviral agent side chain. Bansal et al. (J Pharm Sci. 1981 August; 70(8):850-4 and 855-7) have prepared solid samples of 1,3-dihydroxymethyluracil, 3-hydroxymethyl-1-methyluracil, 1-hydroxymethyl-3-methyluracil, 1-hydroxymethyl-allopurinol, 1,5-dihydroxymethyl-allopurinol, 1-hydroxymethyl-glutethimide, and 1-hydroxymethyl-phenobarbital. The potential usefulness of N-hydroxymethyl compounds as prodrugs was discussed therein. EP 375,329 discloses amino acid esters of pyrimidine and purine nucleosides containing an acyclic side chain, and their use in medical therapy, particularly the treatment and prophylaxis of herpes virus infections. According to the teachings of this patent, the esterification occurs on the hydroxyl groups on the antiviral agent side chain. Rautio et al. (J. Pharmaceut. Sci. 87 (12): 1622-8) teach the preparation of a series of acyloxyalkyl esters of ketoprofen and naproxen, which was aimed at improving the dermal delivery of the drugs. In addition, some hydroxyalkyl esters of ketoprofen were prepared.

Several studies have reported on the synthesis and anticancer activity of N-acyloxyalkyl-5-fluorouracils and N-alkoxycarbonyloxymethyl-5-fluorouracils (Ozaki et al., Chem. Pharm.Bull. Japan 1984, 32, 733-8; Ahmad et al., Chem. Pharm. Bull. 1987, 35, 4137-43; Nagase et al., Heterocycles (1988), 27(5), 1155-8; Nagase et al., Chemistry Letters (1988), (8), 1381-4).

As stated above, 5-FU is an example of one of the most widely used cytotoxic chemotherapeutic agents. However, the most efficient and least toxic route of 5-FU administration is by intravenous bolus or by continuous intravenous infusion, which are both cost-ineffective and patient unfriendly, requiring attendance in hospital or clinic and involving risk of complications from catheterization.

An oral prodrug of 5-FU, tegafur (ftorafur), has therefore been developed. Unfortunately, it was found that upon release of the chemotherapeutic agent itself, it is poorly absorbed and its tissue levels are highly variable, such that a twice-daily and even thrice-daily administration is sometimes necessary. Such a frequent administration is associated with adverse toxic effects and with patient incompliance (Mallet-Martino M, Martino R. Clinical studies of three oral prodrugs of 5-fluorouracil, Oncologist 2002; 7: 288-323). This emphasizes the need for the development of more potent derivatives of 5-FU that will be orally active, will overcome resistance of tumor cells and will required less frequent administration.

Hence, although different approaches have been used heretofore to address the problem of chemotherapeutic drug resistance, including combination drug therapy and the synthesis of various chemical modificators, none of these approaches has proven entirely effective and, in addition, most of these approaches suffered other practical limitations (such as cost, complexity and low yield of synthesis, etc.).

There is thus a widely recognized need for, and it would be highly advantageous to have, novel chemotherapeutic agents devoid of the above limitations and particularly effective in treating conditions associated with drug resistance.

### SUMMARY OF THE INVENTION

According to one aspect of the present invention there is provided a conjugate comprising a first moiety and a second moiety, wherein the first moiety is a chemotherapeutic agent residue and the second moiety is selected such that the conjugate is capable of releasing at least one formaldehyde molecule and/or a formaldehyde analog molecule upon cleavage, with the proviso that neither of the first moiety and the second moiety comprises a psychotropic drug residue.

As is discussed hereinabove, hydroxyalkyl, acyloxyalkyl and alkoxycarbonyloxyalkyl derivatives of 5-fluorouracyl have been described in the art and are therefore excluded from the scope of this aspect of the present invention. However, since the therapeutic beneficiary and efficacy of such compounds have never been taught nor demonstrated hitherto, such conjugates are not excluded from other aspects of the invention.

According to further features in preferred embodiments of the invention described below, the second moiety is selected such that the conjugate is further capable of releasing a biologically active moiety upon cleavage.

According to still further features in the described preferred embodiments the biologically active moiety is selected from the group consisting of a carrier moiety, a therapeutically active moiety, a recognition moiety, and a formaldehyde-releasing group.

According to still further features in the described preferred embodiments the second chemical moiety is selected from the group consisting of a hydroxyalkyl residue, a carboxyalkyl residue, and a first carboxyalkyl residue being covalently linked to a second carboxyalkyl residue.

According to still further features in the described preferred embodiments the first carboxyalkyl residue is covalently linked to the second carboxyalkyl residue via a spacer.

According to still further features in the described preferred embodiments each of the first and second carboxyalkyl residue is capable of releasing formaldehyde.

According to still further features in the described preferred embodiments the chemotherapeutic agent is selected from the group consisting of an anti-cancer agent, an anti-viral agent, an immunomodulating agent, an anti-microbial agent, an anti-mycotic agent, an anti-helminthic agent and an anti-protozal agent.

According to still further features in the described preferred embodiments the chemotherapeutic agent has at least one functional group that is capable of forming a covalent bond with the second moiety.

According to still further features in the described preferred embodiments the functional group is selected from the group consisting of hydroxy, amine and thiol.

According to still further features in the described preferred embodiments the chemotherapeutic agent is selected from the group consisting of acyclovir, abacavir, carmustine, dacarbazine, didanosine, edoxudine, emtricitabine, floxuridine, fludarabine, gangciclovir, gemcitabine, idoxuridine, lamivudin, lomustine, MADU, Nevirapine, Penciclovir, procarbazine, Sorivudine, Stavudine, Tegafur, trifluridine, valaciclovir, zalcitabine, zidovudine, Capecitabine (Xeloda), Thalidomide, Hydroxyurea, Mitomycin C, Vinca, temozolomide, 5-fluorouracil, capecitabine, a nitroso urea, cyclophosphamide, linezolide, penicillin, cephalosporin, sulfa, sulfamethoxazole, fluorocytosine, tolnaftate, caspofungin, thioguanine and 3-mercaptopurine.

According to still further features in the described preferred embodiments the conjugate has the general Formula: 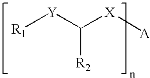
wherein:

n is an integer from 1 to 6;

XnA is a chemotherapeutic agent residue, whereas X is a residue of a functional group that forms a part of the chemotherapeutic agent;

R₂ is selected from the group consisting of hydrogen, alkyl, cycloalkyl and aryl;

Y is O or S; and

R₁ is selected from the group consisting of hydrogen, alkyl, cycloalkyl, aryl and a R₃C(═Z)- group, a (R₃G)₂P(═Z)- group and a R₃S(═Z)₂- group, whereas:

each of Z and G is independently O or S; and

R₃ is selected from the group consisting of hydrogen, alkoxy, arylalkyloxy, alkyl, cycloalkyl, aryl, aminoalkyl, haloalkyl, amine, alkylamine, dialkylamine, carboxyalkyl, carboxyethylene glycol, carboxyether, carboxythioether, ether and thioether.

According to still further features in the described preferred embodiments n is an integer from 1 to 4, preferably from 1 to 2.

According to still further features in the described preferred embodiments X is selected from the group consisting of O, S and NR′, whereas R′ is hydrogen, alkyl, cycloalkyl and aryl.

According to still further features in the described preferred embodiments R₂ is hydrogen.

According to still further features in the described preferred embodiments Y is O.

According to still further features in the described preferred embodiments each of Z and G is O and further, R₁ is R₃C(═O)—.

According to still further features in the described preferred embodiments R₃ is alkyl such as, for example, methyl, ethyl, propyl, butyl, 2-methylpropyl, and tert-butyl.

According to still further features in the described preferred embodiments R₃ is a dicarboxy being conjugated to carboxymethyl.

According to still further features in the described preferred embodiments R₃ is aminoalkyl such as aminopropyl.

Exemplary conjugates according to this and other aspects of the present invention include, but are not limited to:

Butyric acid 5-fluoro-2,4-dioxo-3,4-dihydro-2H-pyrimidin-1-ylmethyl ester;

Butyric acid 5-fluoro-2,6-dioxo-3-(tetrahydro-furan-2-yl)-3,6-dihydro-2H-pyrimidin-1-ylmethyl ester;

Butyric acid 9H-purin-6-ylsulfanylmethyl ester;

Butyric acid 9-butyryloxymethyl-9H-purin-6-ylsulfanylmethyl ester;

Butyric acid 9-butyryloxy-9H-purin-6-ylsulfanylmethyl ester;

Butyric acid 2-(butyryloxymethyl-amino)-9H-purin-6-ylsulfanylmethyl ester;

Butyric acid-9-butyryloxymethyl-2-(butyryloxymethyl-amino)-9H-purin-6-ylsulfanylmethyl ester;

Pentanedioic acid butyryloxymethyl ester 5-fluoro-2,4-dioxo-3,4-dihydro-2H-pyrimidin-1-ylmethyl ester;

Butyric acid (5-fluoro-2,4-dioxo-3,4-dihydro-2H-pyrimidin-1-ylmethoxycarbonylmethoxy)-acetoxymethyl ester;

Butyric acid {2-[2-(5-fluoro-2,4-dioxo-3,4-dihydro-2H-pyrimidin-1-ylmethoxycarbonylmethoxy)-ethoxy]-ethoxy}-acetoxymethyl ester;

Pentanedioic acid butyryloxymethyl ester 5-fluoro-2,6-dioxo-3-(tetrahydro-furan-2-yl)-3,6-dihydro-2H-pyrimidin-1-ylmethyl ester;

Butyric acid [5-fluoro-2,6-dioxo-3-(tetrahydro-furan-2-yl)-3,6-dihydro-2H-pyrimidin-1-ylmethoxycarbonylmethoxy]-acetoxymethyl ester;

Butyric acid [5-fluoro-2,6-dioxo-3-(tetrahydro-furan-2-yl)-3,6-dihydro-2H-pyrimidin-1-ylmethoxycarbonylmethoxy]-acetoxymethyl ester;

5-Fluoro-3-hydroxymethyl-1-(tetrahydro-furan-2-yl)-1H-pyrimidine-2,4-dione;

Diethyl (5-fluoro-2,3-dihydro-3-(tetrahydrofuran-2-yl)-2,6-dioxopyrimidin-1(6H)-yl)methyl phosphate;

2-(1-Butyroyloxymethyl-2,6-dioxopiperidin-3-yl)isoindoline-1,3-dione;

(5-fluoro-2,3-dihydro-3-(tetrahydrofuran-2-yl)-2,6-dioxopyrimidin-1(6H)-yl)methyl4-aminobutanoate;

2-(1-(Hydroxymethyl)-2,6-dioxopiperidin-3-yl)isoindoline-1,3-dione;

2-(1-(Hydroxymethyl)-2,6-dioxopiperidin-3-yl)isoindoline-1,3-dione4-aminobutanoate; and

Diethyl 2-(1-(hydroxymethyl)-2,6-dioxopiperidin-3-yl)isoindoline-1,3-dione phosphate.

According to another aspect of the present invention there is provided a pharmaceutical composition comprising the conjugate described herein and a pharmaceutically acceptable carrier.

According to further features in preferred embodiments of the invention described below, the pharmaceutical composition is packaged in packaging material and identified in print, in or on the packaging material, for use in the treatment of a medical condition selected from the group consisting of proliferative disorder or disease, cancer, an immune-mediated disease, a viral infection or disease, a bacterial infection or disease, a fungal infection or disease, a protozal infection or disease, and a helminthic infection or disease.

According to still further features in the described preferred embodiments the condition is associated with drug resistance.

According to yet another aspect of the present invention there is provided a method of treating a medical condition selected from the group consisting of proliferative disorder or disease, cancer, an immune-mediated disease, a viral infection or disease, a bacterial infection or disease, a fungal infection or disease, a protozal infection or disease, and a helminthic infection or disease, the method comprising administering to a subject in need thereof a therapeutically effective amount of the conjugate described hereinabove, with the only proviso that neither of the first moiety and the second moiety comprises a psychotropic drug residue.

According to still another aspect of the present invention there is provided use of a conjugate as described herein for the treatment of a medical condition selected from the group consisting of proliferative disorder or disease, cancer, an immune-mediated disease, a viral infection or disease, a bacterial infection or disease, a fungal infection or disease, a protozal infection or disease, and a helminthic infection or disease, with the proviso that neither of the first moiety and the second moiety comprises a psychotropic drug residue.

According to an additional aspect of the present invention there is provided use of a conjugate as described herein, for the preparation of a medicament for the treatment of a medical condition selected from the group consisting of proliferative disorder or disease, cancer, an immune-mediated disease, a viral infection or disease, a bacterial infection or disease, a fungal infection or disease, a protozal infection or disease, and a helminthic infection or disease, with the proviso that neither of the first moiety and the second moiety comprises a psychotropic drug residue.

According to still further features in the described preferred embodiments the condition is associated with drug resistance.

According to still further features in the described preferred embodiments the method further comprises administering to the subject a therapeutically effective amount of an additional agent capable of treating the condition.

According to yet an additional aspect of the present invention there is provided a process of preparing the conjugate described herein. The process is effected by providing the chemotherapeutic agent; providing a reactive derivative of the second moiety; and reacting the chemotherapeutic agent and the reactive derivative, thereby providing the conjugate.

According to further features in preferred embodiments of the invention described below, the reacting is performed in the presence of a base.

According to still further features in the described preferred embodiments the process further comprises, prior to the reacting, providing a reactive derivative of the chemotherapeutic agent.

According to still further features in the described preferred embodiments the second moiety and is not a hydroxyalkyl moiety and the reactive derivative of the chemotherapeutic agent is a hydroxyalkyl derivative of the chemotherapeutic agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “therapeutically effective amount” or “pharmaceutically effective amount” denotes that dose of an active ingredient or a composition comprising the active ingredient that will provide the therapeutic effect for which the active ingredient is indicated.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph presenting the IC₅₀ values (average±SEM) of the anti-cancer chemotherapeutic agent Doxorubicin (Dox) in the isogenetic breast carcinoma MCF-7 (resistant) and MCF-7 Dx (sensitive) cell lines;

FIG. 2 is a bar graph presenting the IC₅₀ values (average±SEM) of the anti-cancer chemotherapeutic agent Tegafur and its derivative AN-420 in the isogenetic breast carcinoma MCF-7 (resistant) and MCF-7 Dx (sensitive) cell lines;

FIG. 3 is a bar graph presenting the IC₅₀ values (average±SEM) of the anti-cancer chemotherapeutic agent doxorubicin (Dox) in the isogenetic uterine cancer MES-SA (resistant) and MES-SA DXS (sensitive) cell lines;

FIG. 4 is a bar graph presenting the IC₅₀ values (average±SEM) of the anti-cancer chemotherapeutic agent 6-mercaptopurine and its derivative AN-423 in the isogenetic breast carcinoma MCF-7 (resistant) and MCF-7 Dx (sensitive) cell lines;

FIG. 5 is a bar graph presenting the IC₅₀ values (average±SEM) of the anti-cancer chemotherapeutic agent 6-mercaptopurine and its derivative AN-423 in the isogenetic uterine cancer MES-SA (resistant) and MES-SA DXS (sensitive) cell lines;

FIGS. 6A and 6B present comparative plots demonstrating the effect of Tegafur and its derivative AN-420, according to preferred embodiments of the present invention, on the viability (expressed as % of control) of human resistant (MCF-7, FIG. 6A) and sensitive (MCF-7 Dx, FIG. 6B) breast cancer cells;

FIGS. 7A and 7B are bar graphs presenting the IC₅₀ and IC₉₀ values (average±SEM) of the anti-cancer chemotherapeutic agent tegafur and of derivatives thereof (AN-420 and AN-436), according to the present embodiments, tested in the absence and presence of semicarbazide (SC), in the isogenetic uterine cancer MES-SA (resistant, FIG. 7A) and MES-SA DXS (sensitive, FIG. 7B) cell lines;

FIGS. 8A-8D are bar graphs presenting the IC₅₀ and IC₉₀ values (average±SEM) of the anti-cancer chemotherapeutic agent tegafur and of derivatives thereof (AN-420 and AN-436), according to the present embodiments, tested in the absence and presence of semicarbazide (SC), in human colon carcinoma cells LS-1034 (FIG. 8A) and HT-29 (FIG. 8B), and pancreatic carcinoma cells BXPC-3 (FIG. 8C), and CT-26 (FIG. 8D);

FIG. 9 is a bar graph presenting the IC₅₀ and IC₉₀ values (average±SEM) of AN-438, a derivative of the anti-cancer chemotherapeutic agent thalidomide, according to preferred embodiments of the present invention, in the prostate carcinoma cells 22RV;

FIGS. 10A and 10B present comparative plots demonstrating the effect of thalidomide and its derivative AN-438, according to preferred embodiments of the present invention, on the viability (expressed as % of control) of prostate carcinoma cells 22RV, in the absence or presence of semicarbazide (SC);

FIG. 11 presents comparative plots demonstrating the effect of thalidomide and its derivative AN-438, according to preferred embodiments of the present invention, on the viability (expressed as % of control) of human colon cells HT-29;

FIG. 12 presents comparative plots demonstrating the effect of Tegafur and its derivative AN-420, and of Uracil and its derivative AN-439, according to preferred embodiments of the present invention, on the viability (expressed as % of control) of colon carcinoma cells;

FIGS. 13A and 13B present a bar graph (FIG. 13A) and images (FIG. 13B) demonstrating the effect of Tegafur and its derivative AN-436, according to preferred embodiments of the present invention, on the viability of Lewis lung cancer metastases (as the number of lung lesions);

FIGS. 14A and 14B present comparative plots (FIG. 14A) and a bar graph (FIG. 14B) demonstrating the effect of Tegafur and its derivative AN-436, according to preferred embodiments of the present invention, on the tumor growth (FIG. 14A) and the CEA serum levels (FIG. 14B) of nude mice bearing semicarbazide (SC)-implanted IHT-29 human colon carcinoma cells;

FIG. 15 is a bar graph presenting the effect of Tegafur and its derivative AN-436, according to preferred embodiments of the present invention, on nude mice bearing semicarbazide (SC)-implanted human colon carcinoma cells (LS1034) (expressed as % survival after 82 days); and

FIG. 16 presents the chemical structures of exemplary chemotherapeutic agents that can be utilized to form the conjugates according to the present invention.

### DESCRIPTION OF THE PREFERRED EMBODIMENTS

The present invention is of novel conjugates of chemotherapeutic agents, which are designed so as to release formaldehyde or analogs thereof upon cleavage and thereby to exert enhanced therapeutic activity as compared to that of the chemotherapeutic agent when administered per se. The present invention is further of pharmaceutical compositions containing the novel conjugates of the present invention and their use in the treatment of various medical conditions and particularly in the treatment of medical conditions such as, but not limited to, cancer, viral infections, bacterial infections, fungal infections and other infections, which are characterized by drug resistance. The present invention is further of processes for preparing the novel conjugates described herein.

The principles and operation of the chemical conjugates, the compositions, and the methods, processes and uses according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed in detail in the Background section hereinabove, the use of the presently available chemotherapeutic drugs is oftentimes severely limited by the development of drug resistance thereto, which substantially affects the therapeutic effect of commonly used treatments. Thus, for example, a large number of tumor types develop multidrug resistance to anti-cancer chemotherapeutic agents, rendering these tumors untreatable by the current methods. Similarly, many types of bacteria and other microorganisms develop resistance to the commonly used antibiotics and substantially reduce the efficiency of the current treatment methods. The use of the presently available chemotherapeutic drugs is oftentimes further limited by adverse side effects associated with the administration thereof and therefore more potent agents, which will allow administration of lower doses are required.

Different approaches have been used heretofore to address the resistance associated with chemotherapeutic agents. These include combination drug therapy and use of various chemical modificators, but to date, none of these approaches has proven entirely effective. In addition, most of these approaches suffer other practical limitations, such as cost, complexity and low yield of synthesis, etc.

Interestingly, it has been shown (see, for example, Nudelman et al., J. Med. Chem., 2005, 48, 1047-1054) that formaldehyde, when generated in situ, may contribute to the activity of the chemotherapeutic agent. It has been further shown that the presence of butyric acid and analogous compounds may further provide for enhanced activity of the chemotherapeutic agent. Formaldehyde is known by its activity to kill cells, whereby butyric acid is known to act as histone deacetylase (HDAC) inhibitor which leads to induced cellular death and differentiation.

In fact, WO 02/28345, which is incorporated by reference as if fully set forth herein, discloses methods of enhancing the efficacy of a chemotherapeutic agent by supplementing it with a compound which increases the intracellular levels of endogenous aldehyde. The compounds described in this application are designed to release formaldehyde in situ, and may optionally be designed so as to further release active agents such as butyric acid.

Nevertheless, the compounds taught in WO 02/28345 do not form a part of the chemotherapeutic agent and should be administered in combination therewith, thus imposing complicated formulation and administration routes.

In a search for novel chemotherapeutic agents which would be devoid of the limitations associated with the currently used drugs, and based on the findings delineated above, the present inventors have envisioned that derivatives of chemotherapeutic agents that may release in situ formaldehyde or analogs thereof and may further have another biologically beneficial moiety, may exhibit enhanced therapeutic activity, and thus may circumvent the limitations associated with the presently known drugs.

While reducing the present invention to practice, the present inventors have designed and successfully prepared a novel family of chemical conjugates, which are derivatives of chemotherapeutic agents. These chemical conjugates were designed such that formaldehyde is produced upon their cleavage, optionally along with an additional biologically beneficial moiety, as is detailed hereinbelow. As is demonstrated in the Examples section that follows, such conjugates were easily prepared by derivatizing commonly used chemotherapeutic agents with a chemical moiety, selected such that upon conjugation, a formaldehyde-releasing group is formed. As is further demonstrated in the Examples section that follows, the conjugation method is safe and simple to perform and thus can be applied on a variety of chemotherapeutic agents and generate a variety of aldehyde-releasing groups. As is still further demonstrated in the Examples section that follows, the novel conjugates indeed exhibit increased chemotherapeutical efficacy, and were found particularly efficacious in the treatment of conditions in which drug resistance is exhibited, such as multidrug resistant (MDR) cancer.

Thus, according to one aspect of the present invention there are provided novel chemical conjugates, which are comprised of a first moiety and a second moiety which are covalently linked therebetween. In each of the conjugates described herein, the first moiety is a residue of a chemotherapeutic agent, as is defined hereinbelow, whereby the second moiety is selected such that upon cleavage of the conjugate, one or more molecules of formaldehyde and/or analogs thereof are produced and released. The chemical conjugates are also referred to herein interchangeable as “derivatives of chemotherapeutic agents”.

The phrase “chemotherapeutic agent”, as used herein, includes chemical compounds that exert a therapeutic activity and thus can be used in the treatment of medical conditions. This phrase encompasses compounds that may affect the cell chemistry at different stages of the cell life cycle and of different cells. Thus, this phrase encompasses chemical compounds that may affect, for example, cell growth, cell proliferation, cell viability, and the like, whereby the cells can be, for example, cancer cells, or cells derived from viruses, bacteria, fungi, protozoa, helminthes and the like. This phrase further encompasses immunomodulating agents, as is detailed hereinabove.

This phrase, however, excludes psychotropic drugs.

As used herein, the phrase “psychotropic drug” encompasses any agent or drug that exerts an activity in the central nervous system and thereby can be used in the treatment of various central nervous system diseases or disorders.

Hence, psychotropic drug residues, according to the present invention, include, for example, residues derived from anxiolytic drugs such as, but not limited to, benzodiazepines, phenothiazines and butyrophenones, MAO inhibitors, anti-depressants, anti-convulsive drugs, anti-parkinsonian drugs, and acetylcholine esterase inhibitors. The psychotropic drugs can be tricyclic, bicyclic or monocyclic.

Thus, representative examples of chemotherapeutic agents that are suitable for incorporation within the conjugates of the present invention include, without limitation, anti-cancer agents, antiviral agents including immunomodulators, antimycotic agents (also-referred to herein as anti-fungal agents), antimicrobial agents (also referred to herein as antibiotics or antibacterial agents), anti-protozal agents and anti-helminthic agents, as these agents are described in the Background section hereinabove. The chemotherapeutic agents can also be toxins.

Representative examples of anti-cancer agents that are suitable for use in the context of the present invention include, without limitation, carmustine, dacarbazine, fluxoridine, fludarabine, gemcitabine, lomustine, procarbazine, tegafur, capecitabine (also known as Xeloda), thalidomide, Hydroxyurea, Mitomycin C, vincea, temozolomide, 5-fluoruracil, a nitrosourea, cyclophosamide, thioguanine, cytoxan and 6-mercaptopurine.

Of particular importance in this category are 5-fluoruracil and tegafur, which have been discussed in detail in the Background section hereinabove, and thalidomide. Thalidomide is a derivative of glutamic acid which was first introduced as a sedative and anti-emetic agent for pregnant women in the 1950s. Following reports revealing an association between limb growth defects in babies and maternal usage of thalidomide, this drug was withdrawn from the market in 1961. It was thereafter demonstrated that thalidomide inhibits angiogenesis and can therefore exert anti-cancer activity. It was further found that thalidomide attenuates metastatic potential of tumor cells by reducing TNF-α-induced upregulation of adhesion molecules on endothelial cells such as intracellular adhesion molecule-1 (ICAM-1), vascular-cell adhesion molecule-1 (V-CAM-1), and E-selectins. The fact that thalidomide exerts anti-cancer activity, anti-angiogenetic activity, and also anti-inflammatory activity, render this agent and ant derivative that may enhance its activity, highly potent in treating a myriad of medical conditions.

Representative examples of anti-viral agents that are suitable for use in the context of the present invention include, without limitation, acyclovir, abacavir, didasonine, edoxudine, emtricitabine, floxuridine, lamivudine, idoxuridine, MADU, Nevirapine, Penciclovir, sorivudine, stavudine, trifluridine, valaciclovir, zalcitabine and zidovudine.

Representative examples of anti-microbial agents that are suitable for use in the context of the present invention include, without limitation, linezolide, penicillin, cephalosphorin, sulfa, and sulfamethoxazole.

Representative examples of anti-mycotic agents that are suitable for use in the context of the present invention include, without limitation, fluorocytosine, tolnaftate, and caspofungin.

It should be noted that some of these agents, by treating conditions that may also involve inflammation, may further considered to exert anti-inflammatory activity.

As is discussed hereinabove, the conjugates according to the present invention are chemotherapeutic agents, as defined and described hereinabove, which are derivatized by a chemical moiety, which consists the second moiety. The second moiety is thus covalently attached to the chemotherapeutic agent via a stable, covalent bond, and is selected such that the conjugate is capable of releasing formaldehyde or an analog thereof upon cleavage.

By “cleavage” it is meant herein decomposition of the conjugate, whereby the decomposition can be affected, for example, upon contacting the conjugate with water or an aqueous solution or with air, and/or by in situ metabolism, e.g., by enzymes such as esterases and hydrolases. Preferably, the conjugates of the present invention are designed such that they are readily decompose in situ, namely, upon entering a biological system, tissue or cell, whereby the decomposition can be affected by contacting aqueous fluids or enzymes. The conjugates of the present invention, however, are preferably relatively stable ex-situ, when left intact.

The second moiety and the chemotherapeutic agents are both selected so as to have chemical structures which lead to the presence of a formaldehyde-releasing group within the conjugate.

As used herein, the term “formaldehyde” describes H—C(═O)—H.

The phrase “formaldehyde analog”, as used herein, encompasses structural analogs of formaldehyde, which can be collectively represented by the following general formula:
Ra-C(=D)-Rb

wherein D can be O, S or NR′; and R′, Ra and Rb are each independently hydrogen, alkyl, cycloalkyl, aryl and halo, as these terms are defined herein, provided that at least one of Ra and Rb is hydrogen.

As used herein, the phrase “formaldehyde-releasing group” describes a chemical moiety, which optionally and preferably forms a part of a molecule such as the conjugates described herein, which is capable of releasing (preferably in situ) formaldehyde or formaldehyde analog(s), as defined herein.

The formaldehyde or the analog thereof is released from the formaldehyde-releasing group by decomposition of the group or the molecule of which this group forms a part or by hydrolysis of the group or the molecule of which this group forms a part by, e.g., intracellular esterases or hydrolases.

It is to be understood that the phrase “formaldehyde-releasing group” should be interpreted broadly so as to include moieties that undergo a reaction in situ to form another moiety that is then hydrolysed or decomposes to form formaldehyde.

Moieties that are capable of releasing formaldehyde or analogs thereof in a given environment typically require at least one —CHR— unit, which have appropriate groups immediately adjacent thereto, as are collectively represented by the following general formula:
-Q₁-CHR″-Q₂-

wherein R″ is selected from the group consisting of hydrogen, alkyl, cycloalkyl and aryl, as these terms are defined herein, and Q₁ and Q₂ are each independently selected from the group consisting of carboxy, thiocarboxy, phosphonyl, phosphinyl, phosphoryl, phosphoramide, sulfate, sulfonate, sulfonamide, alkoxy, aryloxy, thioalkoxy, thioaryloxy, amine, hydroxy and thiol, as these terms are defined herein, and optionally one of Q₁ and Q₂ can be hydrogen or halo, as defined herein.

Thus, as noted above, the first and second chemical moieties that compose the conjugates according to the present invention as well as the bond formed by covalently attaching these moieties are selected such that the conjugate includes one or more formaldehyde-releasing group as described herein.

More specifically, the compounds from which the first and second moieties are derived are selected such that each includes at least one functional group that (i) enables the formation of a covalent bond therebetween; and optionally (ii) enables the formation of a formaldehyde releasing group within the thus formed conjugate.

Hence, according to one preferred embodiment of the present invention, the formaldehyde-releasing group is formed upon covalently binding the compounds from which the first and the second moieties are derived. In this case, compounds from which the first and the second moieties are derived are selected such that each includes one or more of a first and a second functional group, respectively, which will allow the formation of one or more covalent bonds therebetween, whereby at least one of these covalent bond forms a part of a formaldehyde-releasing group, as described herein. These compounds can be selected, depending on the functional groups thereof, such that two or more formaldehyde-releasing groups are formed upon conjugation.

Each of the first and the second functional groups present in the compounds from which the first and the second moieties are derived are converted, upon the conjugation, to a residue thereof, whereby the residues form a part of the formaldehyde-releasing group.

Representative examples of functional groups that form a part of the compounds from which the first and the second moieties are derived, which are suitable for use in the context of the present invention therefore include, without limitation, carboxy, thiocarboxy, phosphonyl, phosphinyl, phosphoryl, phosphoramide, sulfate, sulfonate, sulfonamide, alkoxy, aryloxy, thioalkoxy, thioaryloxy, amine, hydroxy, halo, thiol and a reactive methylene derivative thereof, whereby at least one of the first and second functional groups is such a reactive methylene derivative.

As used herein, the phrase “reactive methylene derivative” with respect to the above listed functional groups describes such a functional group that includes and is preferably terminated by a methylene group, as defined herein, that is further substituted by at least one reactive group. Examples of reactive groups include halo, amine, nitro, cyano, nitroaryl, as these terms are defined herein, as any other group that can participate in a conjugation reaction and contribute to the bond formation (including by being a leaving group).

As used herein, the term “methylene” describes a —CR′R″— group, where R′ is as defined hereinabove and R″ is as defined for R′). Preferably, at least one of R′ and R″ is hydrogen and more preferably both R′ and R″ are hydrogen.

Exemplary reactive methylene derivatives include, without limitation, halomethylcarboxy, halomethylsulfonate and the like.

Preferably, the chemotherapeutic agent is selected so as to include a functional group as described hereinabove, whereby the compound from which the second moiety is derived is selected to include a reactive methylene derivative of a functional group.

Thus, preferred chemotherapeutic agents that are suitable for use in the context of the present invention are chemotherapeutic agents that include one or more of the following functional groups: carboxy, thiocarboxy, phosphonyl, phosphinyl, phosphoryl, phosphoramide, sulfate, sulfonate, sulfonamide, alkoxy, aryloxy, thioalkoxy, thioaryloxy, amine, imine, amide, hydroxy, halo and thiol.

While many of the presently known chemotherapeutic agents include one or more functional groups such as amine, hydroxy and thiol, such compounds are the presently most preferred chemotherapeutic agents from which the first moiety in the conjugates is derived according to the present invention.

It should be noted that the amine, hydroxy and thiol groups can be present in the chemotherapeutic agent either per se or can form a part of a ring, e.g., a heteroalicyclic or an heteroaromatic ring, or of a functional group such as amide, imine, ether, thioether, carboxy, thiocarboxy, carbamate, thiocarbamate and the like, as these terms are defined herein.

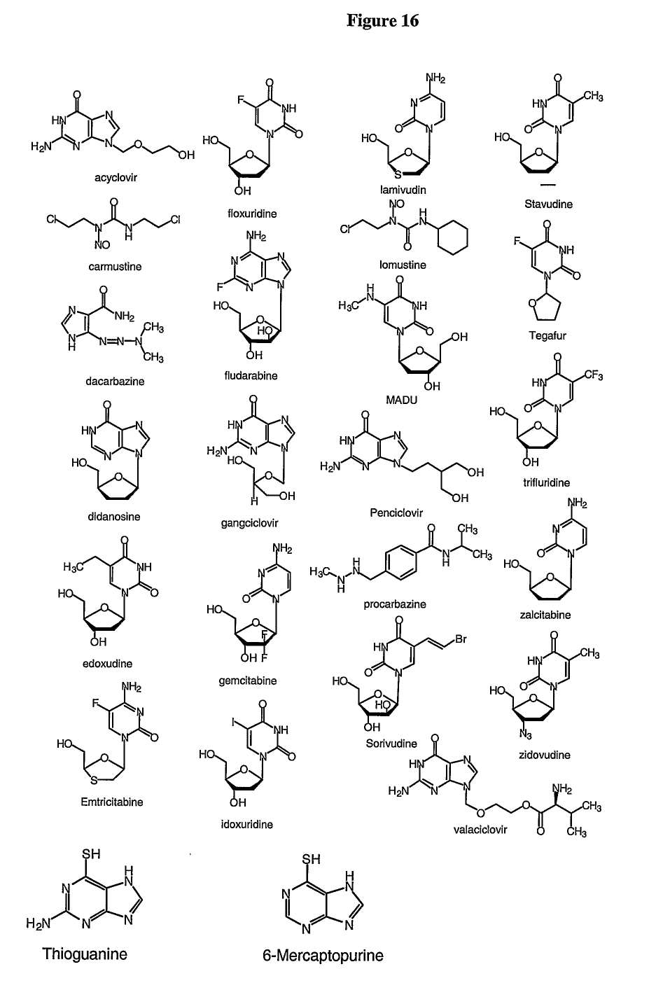

The chemical structures of non-limiting representative examples of preferred chemotherapeutic agents that can be used in that context of the present invention are presented in FIG. 16.

Preferred compound from which the second moiety is derived, according to this embodiment of the present invention, are therefore selected to include one or more functional group as described hereinabove, which is substituted by a reactive methylene group, as described hereinabove.

The compound from which the second moiety is derived can further be selected such that upon decomposition of the compound and releasing one or more formaldehyde molecules or analogs thereof, an additional moiety, which has a beneficial biological or pharmacological activity, is released.

Such an additional moiety, which is also referred to herein interchangeably as a “biological moiety”, can be, for example, a carrier moiety, a recognition moiety, a therapeutically active moiety, and the like. The additional moiety can also be an additional formaldehyde-releasing group. Any combination of these moieties is also within the scope of the present invention.

As used herein, the phrase “carrier moiety” collectively describes a moiety that enhances the bioavailability, solubility and/or biostability of the conjugates and thus provides for a beneficial pharmacological characteristic of the conjugate. Representative examples of carrier moieties that are suitable for use in the context of the present invention include, without limitation, phosphate esters, alkylene glycols such as ethylene glycol and propylene glycol, polyalkylene glycols and the like. The carrier moiety is typically beneficial before the decomposition of the conjugate.

As used herein, the phrase “recognition moiety” describes a moiety that may contribute to the targeting of the conjugate toward the desired location (e.g., tissue, organ or cell) in the body. Recognition moieties are typically designed to include one or more fragments that may recognize and/or bind to a specific receptor or other components on or in the cell or organ where the conjugate exerts its therapeutic activity. Representative examples of recognition moieties include, without limitation, amino acid, peptide, a positively charged group such as guanidine and the like. The specific recognition moiety is preferably selected according to the condition being treated by a selected conjugate according to the present invention. Thus, for example, for treating cancer, the recognition moiety will be selected so as to target the conjugate to the cancerous cells or tissues. For treating a viral or bacterial infection, the recognition moiety will be selected so as to target the conjugate to a membranal receptor or other moiety that is typically present within the virus or bacterium. The recognition moiety preferably includes one part of a binding pair, whereby the other part is present within the cell, tissue or organ to be treated. Representative examples of binding pairs include, without limitation, affinity pairs such as avidin-biotin, enzyme-substrate, antigen-antibody and the like and thus the recognition moiety can be either component of these binding pairs.

As used herein, the phrase “therapeutically active moiety” describes a moiety that may exhibit a therapeutic activity. Preferred therapeutically active moieties that are usable in the context of the present invention include any compound that may exert a therapeutic activity that is beneficial in the context of the condition treatable by the selected conjugate, excluding psychotropic drugs, as defined hereinabove. Thus, for example, when the conjugate includes an anti-cancer chemotherapeutic agent, the therapeutically active moiety can be an anti-proliferative agent, an additional residue of the chemotherapeutic agent that consists the first moiety, a residue of a different anti-cancer chemotherapeutic agent, and the like. When the conjugate includes an anti-bacterial chemotherapeutic agent, the therapeutically active moiety can be an additional residue of the chemotherapeutic agent that consists the first moiety, a residue of a different anti-bacterial chemotherapeutic agent, and the like.

Alternatively, according to another embodiment of the present invention, the compounds from which the first and the second moieties are derived are each selected to include one or more of first and second functional groups, respectively, that allow the formation of a covalent bond therebetween, whereby the formaldehyde-releasing group independently forms a part of the first and/or second moieties and is not formed as a result of the conjugation.

Preferably, the formaldehyde-releasing group forms a part of the second moiety.

Thus, according to this embodiment of the present invention, while the chemotherapeutic agent from which the first moiety is derived preferably includes one or more of the functional groups listed hereinabove, preferred compounds from which the second moiety in the conjugate is derived include a second functional group that may react with the first functional group of the chemotherapeutic agent and an additional moiety that includes a formaldehyde-releasing group, as described hereinabove. The functional group and the formaldehyde-releasing group in the second moiety according to this embodiment of the present invention can be attached one to the other either directly or via a spacer. The spacer can be selected so as to provide the conjugate with a pharmacological beneficial effect such as solubility, bioavailabiliy, biostability and the like. Exemplary spacers include hydrocarbons such as alkyl, alkylene, alkynyl, aryl, cycloalkyl and combinations thereof, optionally interrupted by one or more heteroatoms such as O, S, and/or N, or by one or more additional functional groups such as carboxy, amide, ether, thioether, thiocarboxy, sulfate, sulfonate and the like, alkylene glycols, polyalkylene glycols and the like. Alternatively, the spacer can be a residue of a biologically beneficial moiety such as a recognition moiety or a therapeutically active moiety, as described in detail hereinabove.

Further alternatively, the second moiety can be designed such that it includes two or more formaldehyde-releasing groups, as described hereinabove, whereby one or more formaldehyde-releasing groups are formed by covalently binding the compounds from which the first and second moieties are derived and one or more formaldehyde-releasing groups are included within the second moiety. Such second moieties can be further designed to include a linking moiety, linking the formaldehyde-releasing groups and which is also released upon decomposition of the conjugate. Such a linking group can be selected to provide a beneficial activity, either biological activity, as described above, or a pharmacological activity such as improved solubility and/or stability, as is detailed hereinabove.

Representative examples of second moieties that are suitable for use in this context of the present invention include hydroxyalkyls, which upon cleavage release formaldehyde; and carboxyalkyls, which upon cleavage release formaldehyde and a carboxylic acid. The carboxyalkyl can be further linked to another carboxyalkyl, either directly or via a spacer, as described hereinabove, so as to release two formaldehyde molecules and one or more carboxylic acid moieties.

Preferred conjugates according to the present invention can be represented by the following general formula: 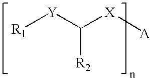
wherein:

n is an integer from 1 to 6;

XnA is a chemotherapeutic agent residue, whereas X is a residue of a functional group that forms a part of said chemotherapeutic agent;

R₂ is selected from the group consisting of hydrogen, alkyl, cycloalkyl and aryl;

Y is O or S; and

R₁ is selected from the group consisting of hydrogen, alkyl, cycloalkyl, aryl and a R₃C(═Z)- group, a (R₃G)₂P(═Z)- group and a R₃S(═Z)₂- group, whereas:

each of Z and G is independently O or S; and

R₃ is selected from the group consisting of hydrogen, alkoxy, arylalkyloxy, alkyl, cycloalkyl, aryl, aminoalkyl, haloalkyl, amine, alkylamine, dialkylamine, carboxyalkyl, carboxyethylene glycol, carboxyether, carboxythioether, ether and thioether.

In this formula, a formaldehyde-releasing group is formed between the first moiety and the second moiety and is represented by the fragment:

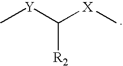

Such a formaldehyde-releasing group is attached to a chemotherapeutic agent residue at one end and to R₁ at the other end.

The first moiety is represented in the general formula above by XnA, whereby X is a residue of the one or more functional groups that are included within the chemotherapeutic agent and serve to covalently bind the second moiety and generate the formaldehyde-releasing group.

As is discussed hereinabove, X is preferably a residue of an amine, hydroxy and thiohydroxy groups, which are present either per se or as a part of heteroalicyclic or heteroaromatic rings, or as a part of functional groups such as amide, carbamates, esters, thioesters, thiocarbamates and the like. Upon conjugation, X in the formula above represents O, NR′ or S, whereby R′ can be hydrogen, alkyl, cycloalkyl or aryl, as these terms are defined herein.

Thus, n is an integer that ranges from 1 to 6, which represents the number of functional groups that are present within the chemotherapeutic agents and to which the second moiety is attached. n therefore further represents the number of formaldehyde-releasing moieties that are formed by the conjugation. Preferably, n is an integer from 1 to 4 and more preferably from 1 to 2.

While the conjugates of the present invention are mainly designed so as to release formaldehyde, preferably, R₂ is hydrogen in the formaldehyde-releasing group is hydrogen. Further preferably, Y is O.

Further preferably, each of Z and G is O, such that the second moiety is a residue of a reactive methylene derivative, as defined hereinabove, of a carboxy, thiocarboxy, a sulfonate or a phosphoryl group, as these terms are defined herein.

In a preferred embodiment of the present invention, R₁ is a residue of a reactive methylene derivative of a carboxy carboxy, and is a R₃C(═O)— group.

R₃ can be an alkyl and preferably a lower alkyl such as methyl, ethyl, propyl, butyl, 2-methylpropyl, and tert-butyl, such that upon decomposition of the conjugate, a carboxylic acid is generated and released. Preferably, the alkyl is propyl and the released carboxylic acid is butyric acid, which, as discussed hereinabove, is a therapeutically active moiety that inhibits histone deacetylase activity. Optionally, the alkyl is an aminoalkyl such as aminopropyl and the released carboxylic acid is GABA, a known therapeutically active moiety.

Alternatively, R₃ is a dicarboxy that is conjugated to a carboxymethyl. Thus, R₃ can include a formaldehyde-releasing group such as carboxymethyl, that is attached to another formaldehyde-releasing group via a dicarboxy spacer.

Representative examples of the conjugates of the present invention that were successfully prepared are presented in Table 1, in the Examples section that follows.

As used herein, the phrase “moiety” describes a residue, as this term is defined hereinbelow, of a chemical substance.

The term “residue”, as used herein, refers herein to a major portion of a molecule, which is covalently linked to another molecule.

As used herein, the term “amine” describes both a —NR′R″ group wherein R′ and R″ are each independently hydrogen, alkyl, cycloalkyl, aryl, as these terms are defined hereinbelow.

The amine group can be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The term “aryl” or “aromatic ring” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The term “nitroaryl” describes an aryl, as defined herein, substituted by one or more nitro groups, as defined herein.

The term “halide” or “halo” describes fluorine, chlorine, bromine or iodine.

The term “haloalkyl” describes an alkyl group as defined above, further substituted by one or more halide.

The term “aminoalkyl” describes an alkyl group, as described above, substituted by one or more amine groups, as defined herein.

The term “aralkyl” describes an alkyl group, as described above, substituted by one or more aryl groups, as defined herein.

The term “sulfate” describes a —O—S(═O)₂—OR′ group, where R′ is as defined hereinabove.

The term “thiosulfate” describes a —O—S(═S)(═O)—OR′ group, where R′ is as defined hereinabove.

The term “sulfonate” describes a —S(═O)₂—R′ group, where R′ is as defined herein.

The term “sulfonamide” describes a —S(═O)₂—NR′R″ group and a R′S(═O)₂—NR″— group, with R′ and R″ as defined herein.

The term “phosphonyl” describes a —P(═O)(OR′)(OR″) group, with R′ and R″ as defined herein.

The term “phosphinyl” describes a —P(═O)(OR′)R″ group, with R′ and R″ as defined herein.

The term “phosphoryl” or “phosphate ester” describes a —P(═O)(OR′)(OR″) group, with R′ and R″ as defined herein.

The term “thiophosphate” or “thiophosphoryl” describes an —O—P(═S)(OR′)(OR″) group, with R′, R″ as defined herein.

The term “phosphoramide” describes a —NR′″—P(═O)(OR′)(OR″) group, with R′ and R″ as defined herein and R′″ as defined for R′ and R″.

The term “hydroxy” or “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” or “thiol” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “nitro” describes an —NO₂ group.

The term “acyl halide” describes a —(C═O)R″″ group wherein R″″ is halide, as defined hereinabove.

The term “carboxy” describes a —OC(═O)R′ group, where R′ is as defined herein.

The term “dicarboxy” describes a R′—C(═O)O-L-O—C(═O)— group, were R′ is as defined herein and L is a spacer such as alkyl, alkenyl, aryl, a heteroalicyclic, heteroaryl, alkylene glycol and the like.

The term “thiocarboxy” describes a —OC(═S)R′ group, where R′ is as defined herein.

The term “carboxyether” describes a R′—C(═O)O-L-O— group, were R′ and L are as defined herein.

The term “carboxythioether” describes a R′—C(═O)O-L-S— group, were R′ and L are as defined herein.

The term “carbamate” describes an —OC(═O)—NR′R″ group, with R′ and R″ as defined herein.

The term “thiocarbamate” describes a —OC(═S)—NR′R″ group, with R′ and R″ as defined herein.

The term “dithiocarbamate” describes a —SC(═S)—NR′R″ group, with R′ and R″ as defined herein.

The term “N-dithiocarbamate” describes an R″SC(═S)NR′— end group or a —SC(═S)NR′— linking group, as these phrases are defined hereinabove, with R′ and R″ as defined herein.

The term “urea”, which is also referred to herein as “ureido”, describes a —NR′C(═O)—NR″R′″ group, where R′ and R″ are as defined herein and R′″ is as defined herein for R′ and R″.

The term “thiourea”, which is also referred to herein as “thioureido”, describes a —NR′—C(═S)—NR″R′″ group, with R′, R″ and R′″ as defined herein.

The term “amide” describes a —C(═O)—NR′R″ group, where R′ and R″ are as defined herein.

The term “guanyl” describes a R′R″NC(═N)— group, where R′ and R″ are as defined herein.

The term “guanidine” describes a —R′NC(═N)—NR″R′″ group, where R′, R″ and R′″ are as defined herein.

The term “hydrazine” describes a —NR′—NR″R′″ group, with R′, R″, and R′″ as defined herein.

The term “heteroaryl” or “heteroaromatic ring” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. Representative examples are pyridine, pyrrole, oxazole, indole, purine and the like.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. Representative examples are piperidine, piperazine, tetrahydrofurane, tetrahydropyrane, morpholino and the like.

The terms “ethylene glycol”, “propylene glycol” and the like, which are collectively referred to herein as “alkylene glycol” describe repeating units of an alkyl (e.g., ethyl, propyl) which are interrupted by oxygen atoms.

The term “polyalkylene glycol” describes a plurality of alkylene glycols, as defined herein, covalently attached therebetween.

The term “imine” describes a —NR′═C—R″ group, where R′ is as described herein.

The term “ether” describes a —R′—O—R″ group, where R′ and R″ are as defiled herein but are not hydrogen.

The term “thioether” describes a —R′—S—R″ group, where R′ and R″ are as defiled herein but are not hydrogen.

According to another aspect of the present invention, there is provided a process of preparing the conjugates described herein. The process is generally effected by providing a chemotherapeutic agent; providing a reactive derivative of the second moiety; and reacting the chemotherapeutic agent and the reactive derivative.

As used herein, the phrase “reactive derivative of the second moiety” describes a compound from which the second moiety is derived and which includes a reactive group, as this phrase is defined herein.

In one embodiment of this aspect of the present invention, the chemotherapeutic agent and the reactive derivative are reacted one with the other, preferably in the presence of a base and/or any agent that can promote the conjugation reaction. Such an agent is selected in accordance with functional groups that form the covalent bonds between the first and the second moieties and thus in accordance with the type of the conjugation reaction.

Conjugation reactions that can be applied in the process according to this aspect of the present invention include commonly known reactions such as nucleophilic reactions, addition-elimination reactions, and the like.

In cases where the formaldehyde-releasing moiety is formed upon the conjugation, the reactive derivative is selected accordingly. Exemplary such reactive groups include, without limitation, the halomethyl derivatives of a carboxy, a phosphoryl, a sulfate or a sulfonate. Such reactive derivatives are suitable for reacting with chemotherapeutic agents that has an amine, hydroxy or thiol functional groups, as described hereinabove.

Alternatively, the reactive derivative can be formaldehyde, which upon conjugation results in the hydroxymethyl derivative of the chemotherapeutic agent.

In another embodiment of this aspect of the present invention, a hydroxyalkyl, preferably hydroxymethyl, derivative of the chemotherapeutic agent is prepared as described herein and is thereafter reacted with the reactive derivative of the second moiety. The reactive derivative of the second moiety in this case can be, for example, a carboxy, an acylhalide and related compounds. The process according to this embodiment is suitable for preparing conjugate in which a formaldehyde-releasing group is included within the second moiety.

As noted hereinabove, exemplary conjugates according to the present embodiments have been readily and efficiently prepared. Detailed exemplary synthetic procedures according to the preferred embodiments of this aspect of the present invention are delineated in the Examples section that follows.

As noted hereinabove, by being capable of releasing formaldehyde or analogs thereof and optionally further releasing a biologically beneficial moiety, the conjugates of the present invention are highly efficacious in the treatment of various conditions, particularly as compared with the efficacy of the non-conjugated chemotherapeutic agents.

The conjugates described herein can therefore be efficiently used in the treatment of various medical conditions such as cancer and other proliferative disorders, immune-mediated diseases, viral infections and diseases, bacterial infections and diseases, fungal infections and diseases, protozal infections and diseases, helminthic infections and diseases, and the like. The conjugates described herein can be further used in the preparation of a medicament for treating such conditions.

Hence, according to another aspect of the present invention there is provided a method of treating such as a medical condition, which is effected by administering to a subject in need thereof a therapeutically effective amount of one or more of the conjugates described herein.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease, substantially ameliorating clinical symptoms of a disease or substantially preventing the appearance of clinical symptoms of a disease.

The term “administering” as used herein refers to a method for bringing a chemical conjugate of the present invention into an area or a site in the brain that affected by the psychotropic disorder or disease.

The term “subject” refers to animals, typically mammals having one or more of the conditions cited above.

The term “therapeutically effective amount” refers to that amount of the conjugate being administered which will relieve to some extent one or more of the symptoms of the condition being treated.

A therapeutically effective amount according to the present invention preferably ranges between about 0.01 mg/kg body and about 100 mg/kg body, more preferably between about 10 mg/kg body and about 100 mg/kg body.

As used herein the term “about” refers to ±10%.

As used herein, the phrase “proliferative disorder or disease” refers to a disorder or disease characterized by cell proliferation. Cell proliferation conditions which may be treated by the conjugates of the present invention include, for example, malignant tumors such as cancer and benign tumors.

As used herein, the term “cancer” refers to various types of malignant neoplasms, most of which can invade surrounding tissues, and may metastasize to different sites, as defined by Stedman's medical Dictionary 25th edition (Hensyl ed., 1990). Examples of cancers which may be treated by the conjugates of the present invention include, but are not limited to, papilloma, blastoglioma, ovarian cancer, prostate cancer, squamous cell carcinoma, astrocytoma, head cancer, neck cancer, bladder cancer, breast cancer, lung cancer, colorectal cancer, thyroid cancer, pancreatic cancer, gastric cancer, hepatocellular carcinoma, leukemia, lymphoma, Hodgkin's lymphoma and Burkitt's lymphoma.

Particular cancers that can be beneficially treated by the conjugates of the present invention include multidrug resistant cancer such as adrenal, colon, jejunal, kidney and liver carcinomas and such cancers that are associated with chromosomal translocation such as AML leukemia, ALL leukemia, Ewing's leukemia.

Other, non-cancerous proliferative disorders are also treatable using the conjugates of the present invention. Such non-cancerous proliferative disorders include, for example, stenosis, restenosis, in-stent stenosis, vascular graft restenosis, arthritis, rheumatoid arthritis, diabetic retinopathy, angiogenesis, pulmonary fibrosis, hepatic cirrhosis, atherosclerosis, glomerulonephritis, diabetic nephropathy, thrombic microangiopathy syndromes and transplant rejection.

The phrase “immune-mediated disease” describes a disease associated with an immune response and which is treatable by immunomodulation, and includes also autoimmune diseases. The phrase “autoimmune disease” describes a disease caused by an immune response such as an autoantibody or cell-mediated immunity to an autoantigen and the like. Representative examples are chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides, dermatomyositis, pemphigus vulgaris, atopic eczema and heparin induced thrombocytopenia.

The phrase “viral infection or disease” describes any infection or disease caused by a virus and include, for example, Herpes Simplex Virus (HSV), Cytomegalovirus (CMV), Human Immunodeficiency Virus (HIV), Hepatitis A, Hepatitis B, Hepatitis C, Herpes, influenza varicella, papilloma, and Klebsiella.

The phrase “bacterial infection or disease” describes an infection or a disease that is caused by a bacterium, and include, for example, mycobacterium tuberculosis, pseudomonas aeruginosa and Mycobacterium lepra.

The phrase “fungal infection or disease” describes an infection or a disease that is caused by a fungus, and include, for example, conditions caused by caused by Candida spp. and Cryptococcus neoformans, Epidermophyton, Trichophyton, and Microsporum.

The phrase “potozal infection” describes an infection or a disease that is caused by a protozeum, and include, for example, entamoeba, giardia, trichomonas, leishmaniasis malaria, toxoplasma, and conditions caused by pneumocystis, trypanosome, rhodesiense and gambiense.

The phrase “helminthic infection” describes an infection or a disease that is caused by a helminth, and include, for example Trichuris trichiura and Ascaris lumbricoides.

It should be noted that some of the conditions that are treatable by the conjugates of the present invention also involve inflammation. Such inflammations are therefore also treatable by the conjugates described herein.

The method, according to this aspect of the present invention, can be further effected by administering to the subject a therapeutically effective amount of an additional active agent that is capable of beneficially affecting the treated condition. These can include any of the known active agents that are used for treating the above-listed conditions. The additional active agent can be administered prior to, concomitant with or subsequent to the admisnitration of the conjugate of the present invention. Optionally, the additional active agent and the conjugate of the present invention can be formulated together and the resulting formulation is administered to the subject.

When utilized for treating the variety of conditions described herein, the conjugates of the present invention can be used either per se or as a part of a pharmaceutical composition.

Thus, further according to the present invention there is provided a pharmaceutical composition which comprises, as an active ingredient, the conjugate described herein and further comprises a pharmaceutically acceptable carrier.

As used herein a “pharmaceutical composition” refers to a preparation of one or more of the conjugates described herein, with other chemical components such as pharmaceutically suitable carriers and excipients. The purpose of a pharmaceutical composition is to facilitate administration of a compound to a subject.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

According to a preferred embodiment of the present invention, the pharmaceutical carrier is an aqueous solution of lactic acid.

In this respect, it should be pointed out that some of the conjugates of the present invention, according to preferred embodiments, are readily soluble in aqueous media and are therefore easily formulated. Such convenient formulation provides an additional advantage of the conjugates of the present invention.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

For injection, the conjugates of the invention may be formulated in aqueous solutions, preferably in physiologically compatible buffers such as Hank's solution, Ringer's solution, or physiological saline buffer with or without organic solvents such as propylene glycol, polyethylene glycol. For transmucosal administration, penetrants are used in the formulation. Such penetrants are generally known in the art.

For oral administration, the conjugates can be formulated readily by combining the active compounds with pharmaceutically acceptable carriers well known in the art. Such carriers enable the conjugates of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carbomethylcellulose and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the conjugates for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The conjugates described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active compound in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the conjugates to allow for the preparation of highly concentrated solutions.

Alternatively, the active ingredient may be in powder form for constitution with a suitable vehicle, e.g., sterile, pyrogen-free water, before use.

The conjugates of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of chemical conjugate effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any conjugate used in the methods of the invention, the therapeutically effective amount or dose can be estimated initially from activity assays in cell cultures and/or animals. For example, a dose can be formulated in animal models to achieve a circulating concentration range that includes the IC₅₀ as determined by activity assays (e.g., the concentration of the test compound, which achieves a half-maximal inhibition of the proliferation activity). Such information can be used to more accurately determine useful doses in humans.

Toxicity and therapeutic efficacy of the chemical conjugates described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active moiety which are sufficient to maintain the psychotropic and/or the anti-proliferative effects, termed the minimal effective concentration (MEC). The MEC will vary for each preparation, but can be estimated from in vitro and/or in vivo data, e.g., the concentration necessary to achieve 50-90% inhibition of a proliferation of certain cells may be ascertained using the assays described herein. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as a FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a conjugate of the invention formulated in a compatible pharmaceutical carrier may also be prepared, placed in an appropriate container, and labeled for treatment of an indicated condition, as is detailed hereinabove.

Hence, according to preferred embodiments of the present invention, the pharmaceutical composition is packaged in a packaging material and is identified in print, on or in the packaging material, for use in the treatment of cancer, a viral infection, a bacterial infection, a fungal infection and the like, as is detailed hereinabove.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples which, together with the above descriptions, illustrate the invention in a non limiting fashion.

### Chemical Syntheses

Materials and Analytical Methods:

Reagents and starting materials were generally purchased from known vendors such as Sigma, Fluka, Aldrich and Merck. Ftorafur, 6-Thioguanine and Uracil were purchased from Sigma; and Thalidomide was purchased from ChemPacific; and were used without further purification.

Silica gel (Merck 60 Art. 9385) was employed for chromatography.

¹H-NMR, ¹³C-NMR, ¹⁹F-NMR, and ³¹P-NMR spectra were obtained on Bruker AC-200, DPX-300 and DMX600 spectrometers. For CDCl₃ and acetone-d₆ solutions, chemical shifts are expressed in ppm downfield from Me₄Si used as internal standard. For D₂O solutions the HOD peak was taken as δ=4.79 (¹H-NMR spectra) or the peak of a small amount of added MeOH taken as δ=49.50 (¹³C-NMR spectra).

Mass spectra were obtained on a Finnigan 4021 spectrometer (CI=chemical ionization, DCI=desorption chemical ionization, EI=electron ionization).

HRMS (high resolution mass spectra) were obtained on a VG AutoSpec E spectrometer. Progress of the reactions was monitored by TLC on silica gel (Merck, Art.5554).

Elemental analyses were conducted using a Thermo Electron Corporation analyzer.

Preparation of N- or S-acyloxyalkyl Derivatives of Chemotherapeutic Agents General Procedure I:

A chemotherapeutic agent (e.g., a purine or pyrimidine analog, 1.53 mmol) was dissolved in a polar solvent (e.g., DMF, 20 ml), and powdered K₂CO₃ (2.75 mmol) was added to the solution thereafter. A chloromethyl ester of a short carboxylic acid (2.29 mmol) in DMF (5 ml) was added dropwise to the resulting solution over 1 hour. The reaction mixture was kept at room temperature for 20 hours, and the formed precipitate was then filtered. The solvent was thereafter removed from the filtrate by evaporation under reduced pressure, and the obtained residue was purified by flash column chromatography on silica gel to give the desired product.

Using the above procedure, exemplary compounds according to the present invention have been prepared, as follows.

Preparation of butyric acid 5-fluoro-2,6-dioxo-3-butyric acid methyl ester 4-hydro-2H-pyrimidin-1-ylmethyl ester (AN-422) and butyric acid 5-fluoro-2,6-dioxo-3,4-dihydro-2H-pyrimidin-1-ylmethyl ester (AN-419): The above compounds were prepared by reacting 5-fluorouracil and chloromethylbutyrate, using a mixture of hexane and ethyl acetate (1:1) as the eluent in the purification procedure. The first fraction gave AN-422 (28% yield) as a viscous oil.

¹H-NMR (CDCl₃): δ=7.72 (d, J=6.25 Hz, 1H, C₆—H), 5.98 (s, 2H, N₃—CH₂O), 5.70 (s, 2H, N₁—CH₂O), 2.38 and 2.33 (two triplets, J=6.25 Hz, 4H, two CH₂CO), 1.68 (two sextets, J=6.25 Hz, 4H, two COCH₂CH₂ CH₃), 0.98 (two triplets, J=6.25 Hz, 6H, two CH₃) ppm.

¹³C-NMR (CDCl₃): δ=173.7 and 172.2 (two CH₂CO), 156.5 (C₄), 149.3 (C₂), 142.0 (C₅), 127.9 (C₆), 70.3 (N₃—CH₂O), 64.7 (N₁—CH₂O), 35.7 and 29.7 (two CH₂CO), 18.2 and 18.1 (two COCH₂CH₂Me), 13.5 (two CH3) ppm.

MS (ES⁺): m/z (%)=331 (MH⁺, 32), 243 (M-C₄O₂H₇, 100), 300 (M-CH₂O, 3).

The second fraction gave AN-419 (37% yield) as white crystals having a melting point of 96-98° C.

¹H-NMR (CDCl₃): δ=7.69 (d, J=6 Hz, 1H, C₆—H), 5.67 (s, 2H, N₁—CH₂O), 2.37 (triplet, J=9 Hz, 2H, CH₂CO), 1.67 (sextet, J=9 Hz, 2H, COCH₂CH₂ CH₃), 0.95 (triplet, J=9 Hz, 3H, CH₃) ppm.

¹³C-NMR (CDCl₃): δ=173.6 (CH₂CO), 157.4 (C₄), 149.5 (C₂), 142.8 (C₅), 128.8 (C₆), 69.6 (N₁—CH₂O), 35.4 (CH₂CO), 17.8 (COCH₂CH₂ CH₃), 13.3 (CH₃) ppm

MS (ES⁺): m/z (%)=231 (MH⁺, 100), 201 (MH⁺—CH₂O, 5).

Preparation of 5-fluoro-2,3-dihydro-3-((R)-tetrahydrofuran-2-yl)-2,6-dioxopyrimidin-1(6H)-yl)methyl butyrate (AN-420): To a solution of tegafur (0.26 gram, 1.31 mmol) in DMF (2 ml) was added K₂CO₃ (0.18 gram, 1.31 mmol) and the mixture was stirred for a few minutes. Chloromethylbutyrate (0.16 ml, 1.31 mmol, 1 equivalent) in 1 ml of DMF was added over 15 minutes. The reaction mixture was stirred for 20 hours at room temperature and was then filtered. To the filtrate were added toluene, water and brine. The organic layer was extracted with 5×15 ml of brine and the aqueous layer was extracted with 5×15 ml of toluene. The organic layers were combined, dried over MgSO₄ and evaporated. The obtained oily residue was dissolved in a minimal amount of ethyl acetate and was then purified by silica gel chromatography, using a mixture of hexane and ethyl acetate (1:3). The filtrate was evaporated and was left to crystallize in the freezer for several hours to give AN-420 (0.33 gram, 83% yield) as off-white crystals having a melting point of 67-68° C.

¹H-NMR (DMSO-d₆): δ=8.05 (d, J=6.9 Hz, 1H, H₁), 5.98 (ddd, J=6.7, 3.4, 1.6 Hz, 1H, H₂), 5.82 (s, 2H, H₆), 4.3+3.86 (dt, J=8, 6 Hz, 1H+q, J=8 Hz, 1H, 2H₅), 2.12+2.01 (m, 1H+m, 1H, 2H₄), 2.32 (t, J=7.1 Hz, 2H, H₇), 1.97 (m, 2H, H₃), 1.56 (sext, J=6.7 Hz, 2H, H₈), 0.9 (t, J=6.7 Hz, 3H, H₉);

¹³C-NMR (DMSO-d₆): δ=171.8 (C₁₃), 156.0 (d, J=26.4 Hz, C₁₁), 148.3 (C₁₂), 139.1 (d, J=228 Hz, C₁₀), 125.0 (d, J=33.2 Hz, C₁), 87.5 (C₂), 69.6 (C₆), 64.2 (C₅), 34.9 (C₇), 31.4 (C₃), 23.4 (C₄), 17.7 (C₈), 13.1 (C₉);

¹⁹F-NMR (DMSO-d₆): δ=166.7 (dd, J=7.2, 1 Hz)

(MS (CI⁺): m/z (%)=301.124 (C₁₃H₁₈N₂O₅F, 100), 231.020 ([M-C₄H₇O]⁺, 62);

HRMS: calculated for C₁₃H₁₈N₂O₅F ([M−1]⁺, DCI/CH₄) 301.119975; found 301.123656.

Elemental Analysis: calculated for C₁₃H₁₇N₂O₅F (300.11) C 52.00, H 5.71, N 9.33; found: C 51.84, H 5.72, N 9.33.

Preparation of butyric acid 9-butyryloxymethyl-2-(butyryloxymethyl-amino)-9H-purin-6-ylsulfanylmethyl ester (AN-425) and Butyric acid 2-(butyryloxymethyl-amino)-9H-purin-6-ylsulfanylmethyl ester (AN-426): the above compounds were prepared by reacting 6-thioguanine and 2.5 mol equivalents of chloromethyl butyrate, using a mixture of hexane and ethyl acetate (1:1) as the eluent in the purification procedure.

The first fraction gave AN-425 (21.5% yield) as a brown oil.

¹H-NMR (CDCl₃): δ=7.97 (s, 1H, CH), 6.00 (two singlets, 4H, NHCH₂O and NCH₂O), 5.97 (s, 2H, SCH₂O), 2.33 (three triplets, J=8 Hz, 6H, three CH₂CO), 1.66 (three sextets, J=8 Hz, 6H, three COCH₂CH₂ CH₃), 0.93 (three triplets, J=8 Hz, 9H, three CH₃) ppm.

¹³C-NMR (CDCl₃): δ=173.2 (three CO), 170.1 (C₁), 158.8 (C₂), 151.4 (C₃), 141.5 (C₄), 136.1 (C₅), 63.7 (SCH₂O), 60.5 (two NCH₂O), 36.0 (three CH₂CO), 18.3 (three COCH₂CH₂ CH₃), 13.6 (three CH₃) ppm.

MS (FAB⁺, Gly): m/z (%)=468 (MH⁺, 22), 378 (MH⁺−3×CH₂O, 100), 207 (MH⁺−3×C₄H₇O₂, 52).

The second fraction gave AN-426 (29% yield) as a yellow oil.

¹H-NMR (CDCl₃): δ=7.97 (s, 1H, CH), 6.00 (s, 2H, NHCH₂O), 5.97 (s, 2H, SCH₂O), 2.33 (two triplets, J=8 Hz, 4H, two CH₂CO), 1.66 (two sextets, J=8 Hz, 4H, two COCH₂CH₂ CH₃), 0.93 (two triplets, J=8 Hz, 6H, two CH₃) ppm.

¹³C-NMR (CDCl₃): δ=173.2 (two CO), 170.1 (C₁), 159.5 (C₂), 151.4 (C₃), 141.5 (C₄), 138.4 (C₅), 63.7 (SCH₂O), 60.2 (NHCH₂O), 36.0 (two CH₂CO), 18.2 (two COCH₂CH₂ CH₃), 13.6 (two CH₃) ppm.

MS (FAB⁺, Gly): m/z (%)=368 (MH⁺, 20), 338 (MH⁺—CH₂O, 35).

Preparation of Butyric acid 9-butyryloxy-9H-purin-6-ylsulfanylmethyl ester (AN-423) and Butyric acid 9H-purin-6-ylsulfanylmethyl ester (AN-421): The above compounds were prepared by reacting 6-mercaptopurine and 1.5 mol equivalents of chloromethyl butyrate, iodomethyl butyrate or 4-nitro-phenoxymethyl butyrate, using a mixture of hexane and ethyl acetate (1:1) as the eluent in the purification procedures. The first fraction gave AN-423 (23.2%, 24%, 23.8% yields respectively) as a yellow oil.

¹H-NMR (CDCl₃): δ=8.85 (s, 1H, C₂—H), 8.30 (s, 1H, C₄—H), 6.23 (s, 2H, NCH₂O), 6.04 (s, 2H, SCH₂O), 2.36 and 2.34 (two triplets, J=8 Hz, 4H, two CH₂CO), 1.68 and 1.66 (two sextets, J=8 Hz, 4H, two COCH₂CH₂ CH₃), 0.94 and 0.92 (two triplets, J=8 Hz, 6H, two CH₃) ppm.

¹³C-NMR (CDCl₃): δ=173.2 (C₁ and two CO), 152.6 (C₄), 149.2 (C₃), 144.1 (C₂), 131.0 (C₅), 63.9 (SCH₂O), 60.3 (NCH₂O), 36.0 and 35.6 (two CH₂CO), 18.2 and 18.0 (two COCH₂CH₂ CH₃), 13.6 and 13.5 (two CH₃) ppm.

MS (ES⁺): m/z (%)=353 (M⁺, 93), 266 (M⁺-C₄O₂H₇, 15), 323 (M⁺-CH₂O, 5).

The second fraction gave AN-421 (31.2%, 33.3%, 33.7% yields respectively) as a yellowish solid.

¹H-NMR (CDCl₃): δ=8.89 (s, 1H, C₂—H), 8.38 (s, 1H, C₄—H), 6.08 (s, 2H, SCH₂O), 2.36 (triplet, J=8 Hz, 2H, CH₂CO), 1.68 (sextet, J=8 Hz, 2H, COCH₂CH₂ CH₃), 0.94 (triplet, J=8 Hz, 3H, CH₃) ppm.

¹³C-NMR (CDCl₃): δ=173.3 (C₁ and CO), 158.3 (C₃), 151.6 (C₄), 149.8 (C₂), 141.9 (C₅), 60.3 (SCH₂O), 35.9 (CH₂CO), 18.2 (COCH₂CH₂ CH₃), 13.5 (CH₃) ppm.

MS (FAB⁺, Gly): m/z (%)=253 (MH⁺, 100), 152 (MH⁺—C₅O₂H₉, 61).

Preparation of (3,4-dihydro-5-methyl-2,4-dioxopyrimidin-1(2H)-yl)methyl pivalate (AN-440) and (2,3-dihydro-5-methyl-2,6-dioxopyrimidin-1(6H)-yl)methyl pivalate (AN-440A): To a 100 ml round flask was added thymine (0.21 gram, 1.7 mmol) in DMF (2 ml). The suspension was stirred until complete dissolution occurred. To the solution was added K₂CO₃ (0.47 gram, 3.38 mmol) and the resulting suspension was stirred for a few minutes, followed by the addition of chloromethyl pivalate (0.48 ml, 3.38 mmol) in DMF (1 ml) over 15 minutes. The mixture was stirred for 20 hours at room temperature and was then filtered. To the filtrate were added toluene, water and brine. The organic layer was extracted with 5×15 ml of brine and the aqueous layer was extracted with 5×15 ml toluene. The organic layers were combined, dried over MgSO₄ and evaporated. The obtained oily residue was purified by silica gel chromatography, using a mixture of hexane and ethyl acetate (5:3). The first fraction gave an oily product, which was subjected to further purification on a column, using a mixture of hexane and ethyl acetate (5:2). The second fraction gave the pure oily bis-product AN-440A (5.9 mg, 16% yield).

¹H-NMR (acetone-d₆): δ=7.66 (q, J=1.5 Hz, 1H, H-1′), 5.90 (s, 2H, H-5′), 5.74 (s, 2H, H-6′), 1.88 (d, J=1.5 Hz, 3H, H-1′), 1.17 (s, 9H, H-8′), 1.15 (s, 9H, H-7′) ppm.

The second fraction gave one of the mono-products AN-440 as white crystals after washings with a solution of n-hexane and diethyl ether (69.8 mg, 17.2% yield).

¹H-NMR (acetone-d₆): δ=10.21 (bs, 1H, H-4), 7.54 (q, J=1.5 Hz, 1H, H-2), 5.68 (s, 2H, H-3), 1.83 (d, J=1.5 Hz, 3H, H-1), 1.18 (s, 9H, H-5) ppm.

Preparation of 2-(1-butroyloxymethyl-2,6-dioxopiperidin-3-yl)isoindoline-1,3-dione (AN-438): To a solution of thalidomide (0.44 g, 1.72mmol) in DMF (6 ml) was added Cs₂CO₃ (0.56 g, 1.72 mmol) and the mixture was stirred for a few minutes until total dissolution. Chloromethyl butyrate (0.23 ml, 1.72 mmol) in 1 ml of DMF was added over 15 minutes. The reaction mixture was then stirred for 20 hours at room temperature. To the mixture were added toluene, water and brine. The organic layer was extracted with 5×15 ml of brine and the aqueous layer was extracted with 5×15 ml of toluene. The organic layers were combined, dried over MgSO₄ and evaporated. The crude white powder was purified by silica gel chromatography, using a mixture of hexane and ethyl acetate (2:1) to give an off-white powder. The powder was dried over P₂O₅ overnight to give AN-438 (0.48 gram, 74% yield). melting point: 132-133° C.

¹H-NMR (DMSO-d₆): δ=7.96-7.89 (m, 4H, AA′BB′), 5.65 (ABq, J=9.8 Hz, 2H, H-4), 5.35 (dd, J=13.2, 5.6 Hz, 1H, H-1ₐₓ), 3.07 (ddd, J=17.6, 13.2, 5.6 Hz, 1H, H-3ₐₓ), 2.84 (ddd, J=17.6, 4.6, 2.9 Hz ,1H, H-3_{eq}), 2.63 (qd, J=13.2, 4.6 Hz, 1H, H-2ₐₓ), 2.26 (triplet, J=7.5 Hz, 2H, H-5), 2.12 (dtd, J=13.2, 5.6, 2.9 Hz, 1H, H-2_{eq}), 1.52 (sextet, J=7.5 Hz, 2H, H-6), 0.87 (triplet, J=7.5 Hz, 3H, H-7) ppm.

¹³C-NMR (DMSO-d₆) δ=171.8 (C-8), 171.1 (C-10), 169.2 (C-9), 167.1 (C-11), 135.0 (C-14), 131.1 (C-12), 123.5 (C-13), 62.9 (C-4), 49.5 (C-1), 35.0 (C-3), 31.0 (C-5), 20.8 (C-2), 17.8 (C-6), 13.3 (C-7) ppm.

MS (ES+): m/z (%)=381 ([M+Na]⁺, 100), 359 (MH⁺, 7.45), 271 ([M-C₄H₇O₂]⁺, 14.51).

Preparation of 5-fluoro-2,3-dihydro-3-(tetrahydrofuran-2-yl)-2,6-dioxopyrimidin-1(6H)-yl)methyl 4-amitiobutanoate trifluoroacetate, (AN-441): To a solution of tegafur (0.456 gram, 2.278 mmol) in DMF (2 ml) was added K₂CO₃ (0.32 gram, 2.28 mmol) and the mixture was stirred for a few minutes. The chloromethyl derivative of Boc-protected GABA (0.57 gram, 2.28 mmol) in 1 ml of DMF was added over 15 minutes. The reaction mixture was stirred for 20 hours at room temperature and was then filtered. To the filtrate were added toluene, water and brine. The organic layer was extracted with ml5×15 ml of brine and the aqueous layer was extracted with ml5×15 ml of toluene. The organic layers were combined, dried over MgSO₄ and evaporated. The obtained oily residue was dissolved in a minimal amount of ethyl acetate and was then purified by silica gel chromatography, using a mixture of hexane and ethyl acetate (1:3). The obtained oily substance was reacted with trifluoroacetic acid (7 equivalents) and stirred for 1 hour. Evaporation of the unreacted acid gave an almost pure yellow oil of the salt AN-441. (0.57 gram, 60% yield).

¹H-NMR (DMSO-d₆) δ=8.05 (d, J=7.0 Hz, 1H, H-1), 7.75 (bs, 3H, H-10), 5.95 (ddd, J=6.5, 3.5, 1.5 Hz, 1H, H-2), 5.81 (ABq system, J=5.9 Hz, 2H, H-6), 4.27+3.81 (dt, J=7.4, 5.6 Hz ,1H, H-5+q, J=7.4 Hz, 1H, H-5), 2.90-2.69 (m, 2H, H-9), 2.49-2.38 (m, 2H, H-7), 2.37-2.13+2.12-2.03 (m, 1H, H-3+m, 1H, H-3), 2.02-1.68 (m, 2H, H-4+m, 2H, H-8) ppm.

¹³C-NMR (DMSO-d₆) δ=171.3 (C-11), 158.5 (q, J=36.9 Hz, C-16), 156.1 (d, J=26.4 Hz, C-12), 148.4 (C-13), 139.2 (d, J=229.4 Hz, C-14), 125.2 (d, J=33.9 Hz, C-1), 115.6 (q, J=290.6 Hz, C-15), 87.6 (C-2), 69.7 (C-6), 64.5 (C-5), 38.1 (C-9), 31.6 (C-3), 30.0 (C-7), 23.5 (C-4), 22.3 (C-8) ppm.

Preparation of (5-fluoro-2,3-dihydro-3-(tetrahydrofuran-2-yl)-2,6-dioxopyrimidin-1(6H)-yl)methyl 2-propylpentanoate (AN-442): To a solution of tegafur (0.46 gram, 2.27 mmol) in DMF (2 ml) was added K₂CO₃ (0.32 gram, 2.28 mmol) and the mixture was stirred for a few minutes. Chloromethyl valeroate (0.44 gram, 2.28 mmol) in 1 ml of DMF was added over 15 minutes. The reaction mixture was stirred for 20 hours at room temperature and was then filtered. To the filtrate were added toluene, water and brine. The organic layer was extracted with ml5×15 ml of brine and the aqueous layer was extracted with ml5×15 ml of toluene. The organic layers were combined, dried over MgSO₄ and evaporated. The obtained oily residue was dissolved in a minimal amount of ethyl acetate and was then purified by silica gel chromatography, using a mixture of hexane and ethyl acetate (1:3) to give AN-442 as an oily substance (0.59 gram, 73% yield).

¹H-NMR (CDCl₃) δ=8.03 (d, J=6.9 Hz, 1H, H-1), 5.95 (ddd, J=6.3, 3.0, 1.5 Hz, 1H, H-2), 5.78 (dABq system, J=9.8, 0.8 Hz, 2H, H-6), 4.27+3.82 (dt, J=8.1, 6.3 Hz, 1H, H-5+q, J=7.4 Hz, 1H, H-5), 2.37-2.23 (m, 2H, H-4, H-3), 2.08-1.98 (m, 2H, H-4, H-7), 1.93 (sextet, J=6.5 Hz, 1H, H-3), 1.50-1.32 (m, 4H, H-8), 1.24 (sextet, J=7.5 Hz, 4H, H-9), 0.82 (triplet, J=7.2 Hz, 6H, H-10) ppm.

¹³C-NMR (DMSO-d₆) δ=174.6 (C-11), 156.0 (d, J=26.4 Hz, C-12), 148.2 (C-13), 139.1 (d, J=235.7 Hz, C-14), 125.1 (d, J=36.9 Hz, C-1), 87.6 (C-2), 69.7 (C-6), 64.3 (C-5), 44.3 (C-7), 34.0 (C-8), 31.6 (C-3), 23.5 (C-4), 19.88+19.86 (2C, C-10) ppm.

¹⁹F-NMR (CDCl₃) δ=−166.7 (ddt, J=6.0, 1.5, 0.75 Hz, 1F).

Preparation of (2,3-Dihydro-2,6-dioxopyrimidin-1(6H)-yl)methyl butyrate (AN-439): To a solution of uracil (0.21 gram, 1.83 mmol) in DMF (3 ml) and acetone (4 ml) was added K₂CO₃ (0.51 gram, 3.66 mmol) and the mixture was stirred for a few minutes. Chloromethyl butyrate (0.5 ml, 3.66 mmol) in 1 ml of DMF was added over 15 minutes. The reaction mixture was stirred for 20 hours at room temperature and was then filtered. To the filtrate were added ethyl acetate and water. The organic layer was extracted with ml5×15 ml of water and the aqueous layer was extracted with ml5×15 ml of ethyl acetate. The organic layers were combined, dried over MgSO₄ and evaporated. The obtained crude white powder was purified by silica gel chromatography, using a mixture of hexane and ethyl acetate (1:1) to give the product (0.12 gram, 31% yield) as a white powder having a melting point of 121-122° C.

¹H-NMR (CDCl₃) δ=8.3 (bs, 1H, NH), 7.51 (d, J=8.4 Hz, 1H, H-1), 5.71 (dd, J=8.4, 2.3 Hz, 1H, H-2), 5.67 (s, 2H, H-4), 2.37 (triplet, J=7.3 Hz, 2H, H-5), 1.66 (sextet, 2H, H-6), 0.94 (triplet, J=7.3 Hz, 3H, H-7) ppm.

¹³C-NMR (CDCl₃) δ=173.8 (C-7), 163.1 (C-8), 150.5 (C-9), 144.6 (C-2), 103.0 (C-1), 69.5 (C-3), 35.8 (C-4), 18.2 (C-5), 13.6 (C-6) ppm.

MS (ES+): m/z (%)=235.0667 ([M+Na]⁺, 100), 251.0351 ([M+K]⁺, 21.55), 213.0880 (MH⁺, 13.35).

Preparation of N- or S-hydroxyalkyl Derivatives of Chemotherapeutic Agents—General Procedure II:

A solution of a chemotherapeutic agent and an aqueous solution of an aldehyde (e.g., formaldehyde, 1.2 mol equivalents) derivative was stirred for 50 minutes in a preheated oil bath at 50° C. The solvent was then evaporated and the obtained residue was dissolved in ethyl acetate and filtered through a silica gel short column. The filtrate was evaporated almost to dryness and diethyl ether was added to crystallize the product.

Using the above procedure, an exemplary compound according to the present invention has been prepared, as follows.

Preparation of 5-fluoro-1-((R)-tetrahydrofuran-2-yl)-3-(hydroxymethyl)pyrimidine-2,4(1H,3H)-dione (AN-436): A solution of tegafur (0.48 gram, 2.4 mmol) and 35% aqueous formaldehyde solution (0.25 ml, 2.9 mmol, 1.2 equivalents), was stirred for 50 minutes in a preheated oil bath at 50° C. The solvent was then evaporated and the colorless residual oil was dissolved in ethyl acetate and filtered through a short silica gel column. The filtrate was evaporated almost to dryness and upon addition of diethyl ether the product crystallized as white crystals (0.46 gram, 83.5% yield) having a melting point of 168-170° C.

¹H-NMR (DMSO-d₆): δ=(7.96 d, J=6.6 Hz, 1H, H-1), 6.43 (triplet, J=7.5 Hz, 1H, H-7), 5.96 (ddd, J=6.3, 3.3, 1.5 Hz, 1H, H-2), 5.20 (d, J=7.5 Hz, 2H H-6), 4.25 (dt, J=7.8, 6.3 Hz, 1H, H-5), 3.82 (dt, J=7.8, 7.2 Hz, 1H, H-5′), 2.26 (m, 1H, H-4), 2.03 (m, 1H, H-4′) 1.93 (m, 2H, H-3) ppm.

¹³C-NMR (DMSO-d₆): δ=156.2 (C-8), 148.7 (C-7), 139.4 (d, J=299.1 Hz, C-9), 124.5 (C-1), 87.3 (C-2), 69.5 (C-6), 64.0 (C-5), 31.6 (C-3), 23.6 (C-4) ppm.

¹⁹F-NMR (DMSO-d₆): δ=−123.69 (ddt, J=7, 1.6, 1 Hz, 1F) ppm.

MS (CI⁺): m/z (%)=229.056 (C₉H₁₀N₂O₄F, 12), 202.0616 ([M-HCO]⁺, 54.81), 131.016 (C₄H₄N₂O₂F⁺, 28.94), (C₄H₈O, 100).

HRMS: calculated for C₉H₁₀N₂O₄F ([M−1]⁺, DCl/CH₄) 229.062460; found 229.056193.

Elemental analysis: calculated for C₉H₁₀N₂O₄F (230.19): C, 46.96; H, 4.82; N, 12.17; found C, 47.12; H, 4.95; N, 12.34.

Preparation of N- or S-phosphonylalkyl Derivatives of Chemotherapeutic Agents—General Procedure III:

To a solution of a freshly distilled N- or S-hydroxyalkyl derivative of a chemotherapeutic agent, prepared according to the general procedure II described above in CH₂Cl₂, freshly distilled pyridine (5 equivalents), dimethylaminopyridine (DMAP, 1 equivalent) and a chlorophosphate (1.3 equivalents) were added. The resulting mixture was stirred at room temperature while being monitored by TLC. Once the reaction was completed, the reaction mixture was extracted with water and ethyl acetate. The organic phases were combined, dried over MgSO₄, and the solvent was evaporated to yield the product.

Using the above procedure, an exemplary compound according to the present invention has been prepared, as follows.

Preparation of diethyl (5-fluoro-2,3-dihydro-3-((R)-tetrahydrofuran-2-yl)-2,6-dioxopyrimidin-1(6H)-yl)methyl phosphate (AN-437): To a solution of N-hydroxymethyl-tegafur (AN-436) (0.23 gram, 0.98 mmol) in CH₂Cl₂ (5 ml), freshly distilled pyridine (0.6 ml, 4.9 mmol, 5 equivalents), DMAP (0.18 gram, 0.98 mmol, 1 equivalent) and diethyl chlorophosphate (0.28 ml, 1.27 mmol, 1.3 equivalent) were added. The resulting mixture was stirred at room temperature while being monitored by TLC (using a mixture of hexane and ethyl acetate 1:1 as an eluent, rf of the starting material=0.1). The reaction mixture was then extracted with water and ethyl acetate. The organic phases were combined, dried over MgSO₄, and the solvent was evaporated. The product was obtained as yellowish oil (0.35 gram, 65% yield).

¹H-NMR (DMSO-d₆): δ=8.04 (d, J=6.6 Hz, 1H, H-1), 5.95 (ddd, J=6.6, 3.6, 1.2 Hz, 1H, H-2), 5.65+5.62 (ddd , J=9.0, 7.0, 0.5 Hz+ddd J=9.0, 5.5, 0.5 Hz, AB system, 2H-6), 4.27+3.83) (dt, J=7.8, 6.0 Hz, 1H, H-5+q, J=7.8 Hz, 1H, H-5), 4.04 (dq, J=7.5, 1.75 Hz, 2H, H-7), 4.03 (dq, J=7.5, 1.75 Hz, 2H, H-9), 2.27+2.04 (dtd, J=13.2, 8.4, 6.6 Hz, 1H, H-4+dddd, J=13.2, 7.2, 6.3, 3.5 Hz, 1H, H-4), 1.93 (tt, J=7.5, 5.5 Hz, 2H, H-3), 1.238 (td, J=6.6, 0.6 Hz, 3H, H-8), 1.236 (td, 7.2, 0.6 Hz, 3H, H-10) ppm.

¹³C-NMR (DMSO-d₆): δ=155.8 (d, J=27 Hz, C-11), 148.2 (C-12), 139.1 (d, J=240 Hz, C-13), 125.1 (d, J=30 Hz, C-1), 87.6 (C-2), 69.7 (C-5), 66.3+63.7 (dd, J=3.0, 0.5 Hz+dd, J=6.5, 1.6 Hz, C-7+C-8), 64.8 (triplet, J=3.0 Hz, C-6), 31.6 (C-3), 23.4 (C-4), 15.8 (d, J=4.0 Hz, 2C-9) ppm.

³¹P-NMR (DMSO-d₆): δ=−2.8 (sept, J=7.9 Hz, 1P) ppm.

MS (ES⁺): m/z (%)=367 (MH⁺, 41.86), 297 ([MH—C₄H₆O]⁺, 100).

Preparation of N- or S-multicarboxyalkyl Derivatives of Chemotherapeutic Agents—General Procedure IV:

To a cold solution (ice bath) of a dicarboxylic acid (1.84 mmol) in dry CH₃CN, under nitrogen atmosphere, N-methyl morpholine (3.38 mmol), 1-(3-dimethylaminopropyl)-3-ethylcarbodiimide HCl (1.84 mmol) and N-hydroxybenzotriazole (HOBT, 1.84 mmol), were added. The solution was stirred for 1 hour at 0° C., a N- or S-hydroxyalkyl derivative of a chemotherapeutic agent, prepared as described in the general procedure II above (1.53 mmol) was added and the resulting mixture stirred for 21 hours at room temperature. CH₃CN was then added (40 ml) and the solution was extracted with 5% NaHCO₃ (×2), H₂O (×2), HCl 1N (×2), dried over MgSO₄ and the solvent was evaporated. The obtained residue was purified by flash column chromatography on silica gel, to give the product in 50-70% yield.

Preparation of pentanedioic acid butyryloxymethyl ester 5-fluoro-2,4-dioxo-3,4-dihydro-2H-pyrimidin-1-ylmethyl ester (AN-427): 1,3-dihydroxymethyl-5-fluorouracil was reacted with an equimolar amount of glutaroyloxymethyl butyrate. The obtained residue was purified by flash column chromatography on silica gel using a mixture of hexane and ethyl acetate (1:1) as the eluent to give the product AN-427 as white crystals (69% yield).

¹H-NMR (CDCl₃): δ=7.68 (d, J=5.5 Hz, 1H, C₃-H), 5.76 (s, 2H, OCH₂O), 5.68 (s, 2H, NCH₂O), 2.47 and 2.44 (two triplets, J=8 Hz, 4H, COCH₂CH₂CH₂CO), 2.35 (triplet, J=8 Hz, 2H, COCH₂CH₂Me), 1.98 (quintet, J=8 Hz, 2H, COCH₂CH₂CH₂CO), 1.67 (sextet, J=8 Hz, 2H, COCH₂CH₂Me), 0.95 (triplet, J=8 Hz, 3H, Me) ppm.

¹³C-NMR (CDCl₃): δ=172.8 (NCH₂OCO), 172.2 (COCH₂CH₂Me), 171.3 (OCH₂OCO), 157.3 (C₁), 149.4 (C₄), 142.5 (C₂), 128.1 (C₃), 79.1 (OCH₂O), 69.8 (NCH₂O), 35.6 (COCH₂CH₂Me), 33.6 and 32.5 (COCH₂CH₂CH₂CO), 24.7 (COCH₂CH₂CH₂CO), 17.9 (COCH₂CH₂Me), 13.3 (Me) ppm.

MS (DCI, CH₄): m/z (%)=375 (MH⁺, 44), 143 (M-C₁₀O₆H₁₅, 35).

Preparation of butyric acid (5-fluoro-2,4-dioxo-3,4-dihydro-2H-pyrimidin-1-ylmethoxycarbonylmethoxy)-acetoxymethyl ester (AN-428): 1,3-dihydroxymethyl-5-fluorouracil was reacted with an equinolar amount of butyric acid carboxymethoxyacetoxymethyl ester. The obtained residue was purified by flash column chromatography on silica gel using a mixture of hexane and ethyl acetate (1:2-1:2.5) as the eluent to give the product AN-428 as white crystals (66% yield).

¹H-NMR (CDCl₃): δ=7.65 (d, J=5.5 Hz, 1H, C₃-H), 5.82 (s, 2H, OCH₂O), 5.76 (s, 2H, NCH₂O), 4.36 and 4.31 (two singlets, 4H, COCH₂OCH₂CO), 2.38 (triplet, J=8 Hz, 2H, COCH₂CH₂Me), 1.68 (sextet, J=8 Hz, 2H, COCH₂CH₂CH₃), 0.98 (triplet, J=8 Hz, 3H, CH₃) ppm.

¹³C-NMR (CDCl₃): δ=172.2 (COCH₂CH₂CH3), 169.8 (NCH₂OCO), 168.4 (OCH₂OCO), 156.8 (C₁), 149.1 (C₄), 141.1 (C₂), 128.4 (C₃), 79.2 (OCH₂O), 70.1 (NCH₂O), 67.8 and 67.7 (COCH₂OCH₂CO), 35.6 (COCH₂CH₂CH₃), 18.0 (COCH₂CH₂ CH₃), 13.5 (Me); MS (FAB⁺, Gly) m/z 377 (MH⁺, 12), 143 (M-C₉O₇H₁₃, 100) ppm.

Preparation of butyric acid {2-[2-(5-fluoro-2,4-dioxo-3,4-dihydro-2H-pyrimidin-1-ylmethoxycarbonylmethoxy)-ethoxy]-ethoxy}-acetoxymethyl ester (AN-429): 1,3-dihydroxymethyl-5-fluorouracil was reacted with an equimolar amount of butyric acid [2-(2-carboxymethoxyethoxy)-ethoxy]-acetoxymethyl ester. The obtained residue was purified by flash column chromatography on silica gel using a mixture of hexane and ethyl acetate (1:3) as the eluent to give the product AN-429 as white crystals (59% yield).

¹H-NMR (CDCl₃): δ=7.68 (d, J=6 Hz, 1H, C₃-H), 5.83 (s, 2H, OCH₂O), 5.73 (s, 2H, NCH₂O), 4.29 (s, 2H, NCH₂OCOCH₂O), 4.22 (s, 2H, OCH₂OCOCH₂O), 3.73 (m, 8H, OCH₂CH₂OCH₂CH₂O), 2.38 (t, J=9 Hz, 2H, COCH₂CH₂CH₃), 1.69 (sextet, J=9 Hz, 2H, COCH₂CH₂ CH₃), 0.99 (t, J=9 Hz, 3H, CH₃) ppm.

¹³C-NMR (CDCl₃): δ=172.3 (COCH₂CH₂CH₃), 170.7 (NCH₂OCO), 169.3 (OCH₂OCO), 157.1 (C₁), 149.2 (C₄), 141.8 (C₂), 128.7 (C₃), 79.1 (OCH₂O), 71.0 (NCH₂O), 70.9 (C₇), 70.6 (C₆), 70.5 (C8), 69.9 (C₅), 68.2 (two COCH₂O), 35.7 (COCH₂CH₂CH₃), 18.2 (COCH₂CH₂ CH₃), 13.6 (CH₃) ppm.

MS (FAB⁺, Gly) ): m/z (%)=465 (MH⁺, 25), 144 (MH⁺—C₁₃O₉H₂₁, 100).

Preparation of Pentanedioic acid butyryloxymethyl ester 5-fluoro-2,6-dioxo-3-(tetrahydro-furan-2-yl)-3,6-dihydro-2H-pyrimidin-1-ylmethyl ester (AN-430): 5-Fluoro-1-((R)-tetrahydrofuran-2-yl)-3-(hydroxymethyl) pyrimidine-2,4(1H,3H)-dione (AN-436) was reacted with an equimolar amount of glutaroyloxymethyl butyrate. The obtained residue was purified by flash column chromatography on silica gel using a mixture of hexane and ethyl acetate (1:1) as the eluent to give the product AN-430 as white solid (58% yield).

¹H-NMR (CDCl₃): δ=7.46 (d, J=5.5 Hz, 1H, C₆—H), 5.98 (m, 1H, C₁—H), 5.94 (s, 2H, NCH₂O), 5.75 (s, 2H, OCH₂O), 4.25 and 4.02 (two multiplets, 2H, C₄-2H), 2.47 and 2.44 (two triplets, J=8 Hz, 4H, COCH₂CH₂CH₂CO), 2.46 and 2.11 (two multiplets, 2H, C₂-2H), 2.35 (t, J=8 Hz, 2H, COCH₂CH₂CH₃), 1.98 (quintet, J=8 Hz, 2H, COCH₂CH₂CH₂CO), 1.94 (m, 2H, C₃-2H), 1.67 (sextet, J=8 Hz, 2H, COCH₂CH₂ CH₃), 0.95 (t, J=8 Hz, 3H, CH₃) ppm.

¹³C-NMR (CDCl₃): δ=171.4 (three CH₂CO), 156.5 (C₈), 148.5 (C₇), 142.0 (C₅), 122.4 (C₆), 88.3 (C₁), 79.2 (OCH₂O), 70.4 (NCH₂O), 64.5 (C₄), 35.8 (COCH₂CH₂ CH₃), 33.0 and 32.8 (COCH₂CH₂CH₂CO), 32.7 (C₂), 23.8 (C₃), 19.6 (COCH₂CH₂CH₂CO), 18.1 (COCH₂CH₂ CH₃), 13.5 (CH₃) ppm.

MS (FAB⁺, Gly): m/z (%)=445 (MH⁺, 6), 213 (MH⁺—C₁₀O₆H₁₅, 16)

Preparation of butyric acid [5-fluoro-2,6-dioxo-3-(tetrahydro-furan-2-yl)-3,6-dihydro-2H-pyrimidin-1-ylmethoxycarbonylmethoxy]-acetoxymethyl ester (AN-431): AN-436 was reacted with an equimolar amount of butyric acid carboxymethoxyacetoxymethyl ester (. The obtained residue was purified by flash column chromatography on silica gel using a mixture of hexane and ethyl acetate (1:1) as the eluent to give the product as a white solid (54% yield).

¹H-NMR (CDCl₃): δ=7.47 (d, J=5.5 Hz, 1H, C₆—H), 6.06 (s, 2H, NCH₂O), 5.99 (m, 1H, C₁—H), 5.83 (s, 2H, OCH₂O), 4.30 and 4.28 (two singlets, 4H, COCH₂OCH₂CO), 4.25 and 4.04 (two multiplets, 2H, C₄-2H), 2.49 and 2.11 (two multiplets, 2H, C₂-2H), 2.38 (t, J=8 Hz, 2H, COCH₂CH₂ CH₃), 1.98 (m, 2H, C₃-2H), 1.68 (sextet, J=8 Hz, 2H, COCH₂CH₂ CH₃), 0.98 (t, J=8 Hz, 3H, Me) ppm.

¹³C-NMR (CDCl₃): δ=172.2 (COCH₂CH₂Me), 168.5 (C₈), 168.4 (COCH₂OCH₂CO), 153.3 (C₇), 141.0 (C₅), 128.8 (C₆), 88.3 (C₁), 79.2 (OCH₂O), 75.7 (NCH₂O), 67.7 and 67.6 (COCH₂OCH₂CO), 64.7 (C₄), 35.7 (COCH₂CH₂ CH₃), 29.7 (C₂), 23.8 (C₃), 18.0 (COCH₂CH₂ CH₃), 13.5 (CH₃) ppm.

MS (FAB⁺, Gly): m/z (%)=447 (MH⁺, 62), 343 (MH⁺-2XCH₂O—CO₂, 100), 417 (MH⁺—CH₂O, 46), 213 (M-C₉O₇H₁₃, 58).

Preparation of butyric acid (2-{2-[5-fluoro-2,6-dioxo-3-(tetrahydro-furan-2-yl)-3,6-dihydro-2H-pyrimidin-1-ylmethoxycarbonylmethoxy]-ethoxy}-ethoxy)-acetoxymethyl ester (AN-432): 5-Fluoro-1-((R)-tetrahydrofuran-2-yl)-3-(hydroxymethyl)pyrimidine-2,4(1H,3H)-dione (AN-436) was reacted with an equimolar amount of butyric acid [2-(2-carboxymethoxyethoxy)-ethoxy]-acetoxymethyl ester. The obtained residue was purified by flash column chromatography on silica gel using a mixture of hexane and ethyl acetate (1:3) as the eluent to give the product AN-432 as viscous oil (51% yield).

¹H-NMR (CDCl₃): δ=7.47 (d, J=5.5 Hz, 1H, C₆—H), 6.01 (s, 2H, NCH₂O), 5.99 (m, 1H, C₁—H), 5.81 (s, 2H, OCH₂O), 4.25 and 4.03 (two m's, 2H, C₄-2H), 4.21 (s, 2H, NCH₂OCOCH₂O), 4.18 (s, 2H, OCH₂OCOCH₂O), 3.76 (m, 8H, OCH₂CH₂OCH₂CH₂O), 2.47 and 2.11 (two m's, 2H, C₂-2H), 2.36 (t, J=8 Hz, 2H, COCH₂CH₂Me), 1.96 (m, 2H, C₃-2H), 1.68 (sextet, J=8 Hz, 2H, COCH₂CH₂ CH₃), 0.98 (t, J=8 Hz, 3H, CH₃) ppm.

¹³C-NMR (CDCl₃): δ=172.1 (COCH₂CH₂Me), 169.2 (OCH₂OCOCH₂O), 168.5 (NCH₂OCO and C₈), 153.3 (C₇), 141.0 (C₅), 128.8 (C₆), 88.4 (C₁), 79.2 (OCH₂O), 75.7 (NCH₂O), 71.4 (C₁₁), 71.1 (C₁₀), 70.9 (C₁₂), 70.3 (C₉), 68.7 (OCH₂OCOCH₂O), 68.2 (OCH₂COOCH₂N), 64.7 (C₄), 35.7 (COCH₂CH₂ CH₃), 29.7 (C₂), 23.8 (C₃), 18.0 (COCH₂CH₂ CH₃), 13.5 (CH₃) ppm.

MS (FAB⁺, Gly)): m/z (%)=535 (MH⁺, 5), 213 (M-C₁₃O₉H₂₁, 22).

Other chemotherapeutic agent derivatives according to the present embodiments were similarly prepared.

Table 1 below presents the chemical structures and names of some exemplary compounds which were prepared and utilized in the activity assays described herein under according to the present invention

TABLE 1

| | General | | |
|---|---|---|---|
| Code | Procedure | Chemical Name | Structure |
| AN419 | I | Butyric acid 5-fluoro-2,6- dioxo-3,4-dihydro-2H- pyrimidin-1-ylmethyl ester | 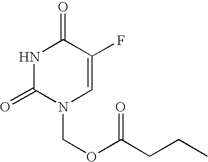 |
| AN-420 | I | Butyric acid 5-fluoro-2,6- dioxo-3-(tetrahydro-furan- 2-yl)-3,6-dihydro-2H- pyrimidin-1-ylmethyl ester | 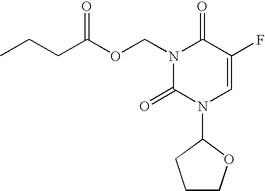 |
| AN-421 | I | Butyric acid 9H-purin-6- ylsulfanylmethyl ester | 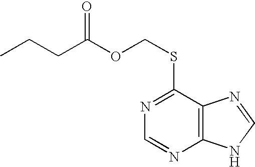 |
| AN-422 | I | Butyric acid 5-fluoro-2,6- dioxo-3-Butyric acid methyl ester 4-hydro-2H- pyrimidin-1-ylmethyl ester | 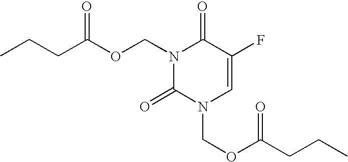 |
| AN-423 | I | Butyric acid 9-butyryloxy- 9H-purin-6- ylsulfanylmethyl ester | 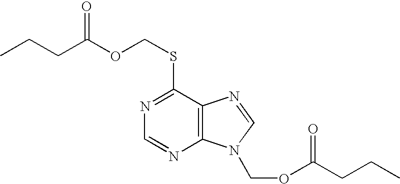 |
| AN-425 | I | Butyric acid 2- (butyryloxymethyl-amino)- 9H-purin-6- ylsulfanylmethyl ester | 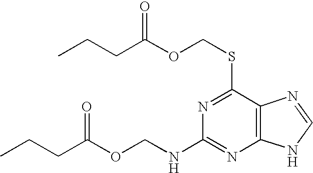 |
| AN-426 | I | Butyric acid-9- butyryloxymethyl-2- (butyryloxymethyl-amino)- 9H- purin-6-ylsulfanylmethyl ester | 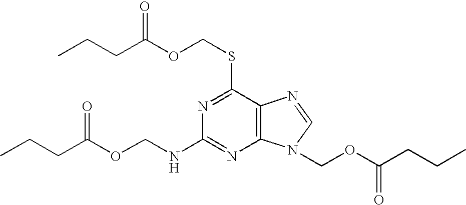 |
| AN-427 | IV | Pentanedioic acid butyryloxymethyl ester 5- fluoro-2,4-dioxo-3,6- dihydro-2H-pyrimidin-1- ylmethyl ester | 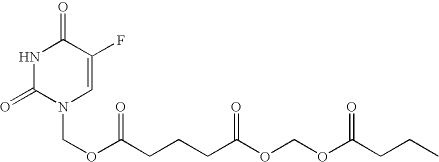 |
| AN-428 | IV | Pentanedioic acid butyryloxymethyl ester acetic acid 5-fluoro-2,4- dioxo-3,6-dihydro-2H- pyrimidin-1-acetoxymethy ester | 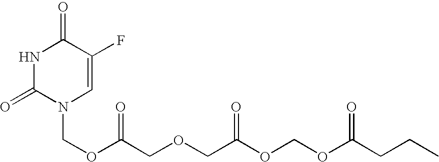 |
| AN-429 | IV | Butyric acid {2-[2-(5- fluoro-2,4-dioxo-3,6- dihydro-2H-pyrimidin-1- ylmethoxycarbonylmethox)- ethoxy]-ethoxy}- acetoxymethyl ester | 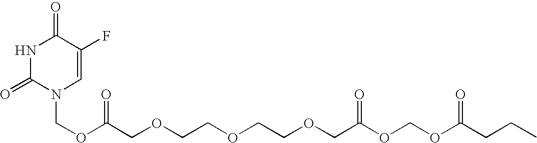 |
| AN-430 | IV | Pentanedioic acid butyryloxymethyl ester 5- fluoro-2,6-dioxo-3- (tetrahydro-furan-2-yl)-3,6- dihydro-2H-pyrimidin-1- ylmethyl ester | 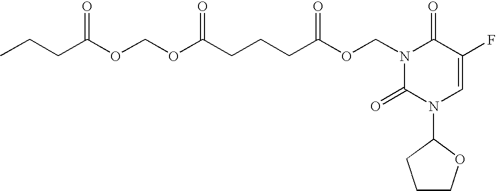 |
| AN-431 | IV | Butyric acid [5-fluoro-2,6- 2-yl)-3,6-dihydro- 2H-pyrimidin- 1-ylmethoxy- carbonylmethoxy]- acetoxymethyl ester | 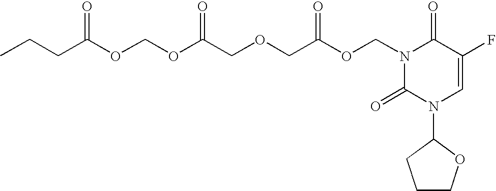 |
| AN-432 | IV | Butyric acid (2-{2-[5- fluoro-2,6-dioxo-3- (tetrahydro-furan-2-yl)4- hydro-2H-pyrimidin-1- ylmethoxycarbonylmethoxy]- ethoxy}-ethoxy)- acetoxymethyl ester | 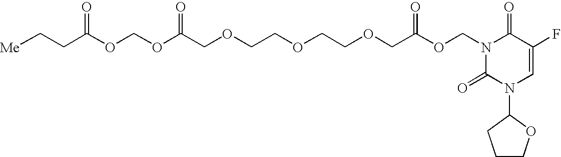 |
| AN-436 | II | 5-Fluoro-3-hydroxymethyl- 1-(tetrahydro-furan-2-yl)- 1H-pyrimidine-2,4-dione | 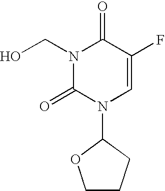 |
| AN-437 | III | Diethyl (5-fluoro-4-hydro- 3-(tetrahydrofuran-2-yl)- 2,6-dioxopyrimidin-1(6R)- yl)methyl phosphate | 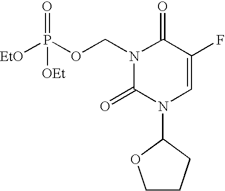 |
| AN-438 | I | 2-(1-Butyroyloxymethyl- 2,6-dioxopiperidin-3- yl)isoindoline-1,3-dione | 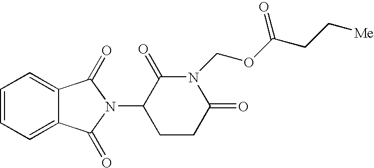 |
| AN-439 | I | (5-fluoro-4-hydro-3- (tetrahydrofuran-2-yl)-2,6- dioxopyrimidin-1(6R)- yl)methyl 4- aminobutanoate | 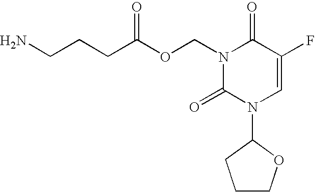 |
| AN-440 | I | (3,4-Dihydro-5-methyl-2,4- dioxopyrimidin-1(2H)- yl)methyl Pivalate | 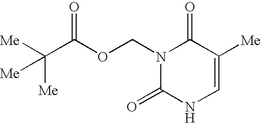 |
| AN-441 | I | (5-Fluoro-2,3-dihydro-3- (tetrahydrofuran-2-yl)-2,6- dioxopyrimidin-1(6H)- yl)methyl 4- aminobutanoate trifluoroacetate | 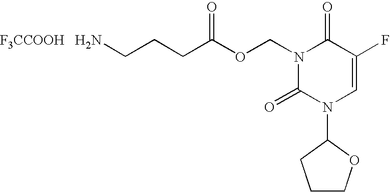 |
| AN-442 | I | 5-Fluoro-2,3-dihydro-3- (tetrahydrofuran-2-yl)-2,6- dioxopyrirnidin-1(6H)- yl)methyl 2- propylpentanoate | 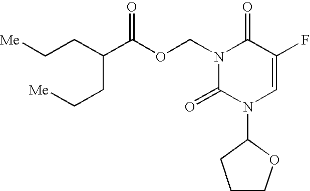 |
| AN-443 | II | 2-(1-(Hydroxymethyl)-2,6- dioxopiperidin-3- yl)isoindoline-1,3-dione | 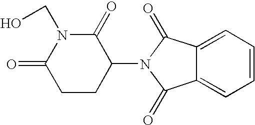 |
| AN-444 | III | Diethyl 2-(1- (Hydroxymethyl)-2,6- dioxopiperidin-3- yl)isoindoline-1,3-dione phosphate | 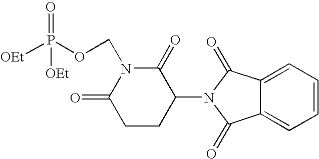 |
| AN-445 | III | 2-[1- (4Aminobutyroyl)oxymethyl- 2,6-dioxopiperidin-3- yl)isoindoline-1,3-dione | 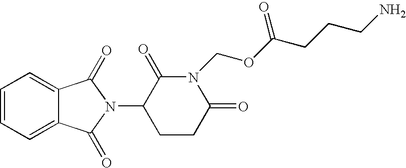 |
| AN-446 | I | 2,3-Dihydro-2,6- dioxopyrimidin-1(6H)- yl)methyl butyrate | 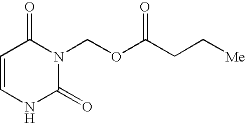 |

### Activity Assays

Materials and Experimental Methods:

In Vitro Studies:

Cells: The human breast cancer cell lines MCF-7 and MCF-7 DX, were obtained from Dr. R. Supino (Laboratorio Oncologia, Milano, Italy); the murine 3LLD122 highly metastatic Lewis lung carcinoma cell line was obtained from Dr. Eisenbach L, the Weizmann Inst., Israel. The following cell lines were purchased from the ATCC, USA: CT-26, an N-nitroso-N-methylurethane-(NNMU) induced murine (BALB-C) colon carcinoma; Panc02, a mouse (C57/Bl) pancreas adenocarcinoma; HT-49, a human colorectal adenocarcinoma; LS1034, a multidrug resistant human colorectal carcinoma; BxPC-3 human pancreas adenocarcinoma; MES-SA and MES-SA-DXS, human uterine sarcoma sensitive and multidrug resistant, respectively; and Human prostate carcinoma 22RV1, a subclone of CWR22 cells.

The cells were maintained in DMEM growth medium supplemented with 10% heat inactivated fetal calf serum, 2 mM glutamine, penicillin (250 units/ml) and streptomycin (125 μg/ml) in a humidified 5% CO₂/air atmosphere at 37° C.

Proliferation assay: cells at a density of 3-5×10⁴ cells/ml in 200 μl medium supplemented with 10% FCS were seeded in tissue culture 96-well plates for 24 hours, and were then exposed for 72 hours to a novel derivative of a chemotherapeutic agent (in quadruplicate) as specified. After 3 days, the cells were rinsed with PBS, and fixed with ethanol (70%) for 30 minutes. The ethanol was thereafter discarded, 200 μl of 10 μg/ml HOECHST fluorescent dye (Sigma) solubilized in PBS were added and the fluorescence was measured at 390-460 nm with a FluoStar fluorometer. MTT solution (10 ml of 5 mg/ml MTT (Sigma) in PBS) was added, and the plate was incubated for 3 hours. Formazan crystals were dissolved with 140 ml of 0.04 HCl-isopropanol. The optical density of the wells was measured with a microplate reader at 540 nm.

Apoptosis assay: Cells (2×10⁵ cells/ml) were seeded in 24-well plates and incubated with a derivative of a chemotherapeutic agent according to the present embodiments, as specified, for 24 hours. The cells were thereafter collected and double-stained with annexin V-FITC and propidium iodide, according to the manufacturer's instructions (MBL, Japan). Alive, apoptotic and total dead cells were quantitated by flow cytometry using a FACS calibur cytometer (Becton Dickinson).

In Vivo Studies:

All animal experiments were conducted according to the NIH Laboratory Animal Care Guidelines and in accordance with the approval of the Tel Aviv University Committee for Animal Experimentation.

Xenograft flank colon carcinoma models: Eight weeks old, male CD1 nude mice (Harlan, Israel) were inoculated sc in the flanks with 5×10⁶ HT29 or LS1034 cells. When tumor volume reached 50-100 mm³, treatment with derivatives of chemotherapeutic agents according to the present embodiments commenced and was given by gavage five times a week for the duration of the experiment at the specified doses. Tumor volume (determined by caliper) and body weights were measured twice weekly. Tumor volume was measured (mm) using the formula for an ellipsoid sphere:
Length×Width²/2=Volume (mm)³

This commonly used formula was also used to calculate tumor weight, assuming unit density (1 mm³=1 mg).

Xenograft flank of human prostate model: Male CD1 nude mice (Harlan, Israel) were inoculated sc in their flank with 107 22Rv1 cells, randomized, divided into two groups (at least 12 animals per group) and treated, 24 hours after cell implantation, with vehicle alone (untreated control) or with various doses of a derivative of a chemotherapeutic agent according to the present embodiments (25-100 mg/kg), given by gavage daily five times a week. Mice were weighed and tumors, which developed in more than 90% of the animals, were measured twice a week with a caliper as described above.

Xenograft orthotopic prostate carcinoma model: Ten weeks old male CD1 nude mice were anesthetized with an intraperitoneal injection of kitamine hydrochloride and xylazine. A transverse incision was made in the lower abdomen, and the bladder and seminal vesicles were delivered through the incision to expose the dorsal prostate. 22RV1 cells (5×10⁵ cells/50 μl PBS) were injected under the prostatic capsule by means of a 27-gauge needle. The incision was closed using a running suture of 5-0 silk.

Metastatic lung model: Female C57/BL mice, 6-10 weeks (Harlan, Israel) were intraventricularly implanted (in the tail vein) with 5×10⁵ 3LLD122 cells. Treatment with a derivative of a chemotherapeutic agent according to the present embodiments, for five days per week, commenced 24 hours post cell implantation. After 21 days the animals were sacrificed and their lung lesions were scored under an illuminated magnifying glass.

Antibodies: Polyclonal rabbit anti-human Bax Ab (Santa Cruz, Calif., USA); polyclonal rabbit anti-human prostate specific antigen (PSA) Ab (DakoCytomation, Glostrup, Denmark); polyclonal rabbit anti-human Her-2/neu (NeoMarkers, Fremont, Calif., US); monoclonal rat anti mouse CD34 (Accurate Chemicals & Scientific, Westbury, N.Y., US); and polyclonal rabbit anti-human pan CEA (Santa Cruz, Calif., USA); were used for immunohistochemistry (IHC) and Western blot analysis. Horseradish peroxidase (HRP)-conjugated goat anti-rabbit IgG, HRP-conjugated goat anti-mouse IgG (Jackson ImmunoResearch Laboratories, USA); biotin goat anti-rabbit IgG (Santa Cruz, Calif., USA) were the secondary antibodies for all the described procedures.

Immunohistochemistry staining of paraffin-embedded blocks: Tumors from sacrificed mice were fixed for 24 hours in 4% paraformaldehyde and dehydrated in increasing alcohol concentrations (from 70% to 100%). Paraffin blocks were prepared and 5 μm sections were deparaffinized. Endogenous peroxidase activity was inhibited by incubation of the sections in 1% hydrogen peroxide. The sections were blocked with 5% FCS, stained with either CEA or bFGF (diluted 1:200) followed by HRP-conjugated goat anti-rabbit IgG secondary antibody reacted with DAB or nickel DAB (Vector, USA), counterstained with hematoxylin and dehydrated. Slides were examined using an Olympus B ×52 light microscope and images were taken with a digital camera.

Serum PSA or CEA: Blood taken from mice was allowed to coagulate, centrifuged and the collected serum was analyzed for PSA with an ELISA kit (Diagnostic Systems Laboratories, Webster, Tex., USA) or CEA kit (MP Biomedical, Orangburg, N.Y.). At the end of the experiment blood samples were drawn from the eyes of mice (anesthetized slightly by inhalation of ether) or from the hearts of sacrificed animals.

Western blot analysis: Whole cell tissue lysates or acid extracted histones were solubilized in a 4-fold concentrated sample buffer and 1-30 mg protein samples were separated by SDS-PAGE. The proteins were transferred to a nitrocellulose membrane (Schleicher & Schuell, Germany) and probed with the primary antibodies as indicated, followed by incubation with an HRP-goat anti-rabbit IgG secondary antibody. Visualization of the bound HRP-goat anti-rabbit IgG was performed using the enhanced chemiluminescence that was done using Western Blotting Luminol reagent (cat No. SC-2048, Santa Cruz, Calif., US). Equal amounts of reagent A and B were mixed, and added to the nitrocellulose membrane for 1 minute. Excess liquid was absorbed and the membrane was exposed for 5 minutes to Chemiluminescence BioMax light film (cat. No. 868 9358, Kodak, USA). The intensity of the bands was measured with a Versa Doc instrument with Quantityl program (Bio-Rad, USA). The fold increase of each specific protein was determined by the ratio of band intensities of treated and untreated cells, normalized to loading control (actin or histone H3).

Treatments and schedules: Table 2 below summarizes the animals number and characteristics in each of the tested experimental models described above

TABLE 2

| Group | | No. | | Age |
|---|---|---|---|---|
| No. | Model | Animals | Sex | (weeks) |
| 1 | colon carcinoma models | >12 | Male | 6-8 |
| 2 | human prostate model | 10 | Male | 6-8 |
| 3 | orthotopic prostate carcinoma | 10 | Male | 10 |
| | model | | | |
| 4 | Metastatic lung model | 10 | Female | 6-10 |

Data Analysis:

IC₅₀ and IC₉₀ values were determined from the best-fitted curve where the percentages of survival were 50 or 90 respectively.

The statistical significance of the effects in comparative studies was determined by Student's t-test.

Comparisons of chemotherapeutic agent activity were done by t-test.

Survival was estimated by the Kaplan-Meier curves, and the difference in survival between the groups was analyzed by Wilcoxon's test and using a log-rank test chi-square (JMP 5.0.1.2, Chicago, Ill.).

### Experimental Results

In Vitro Studies:

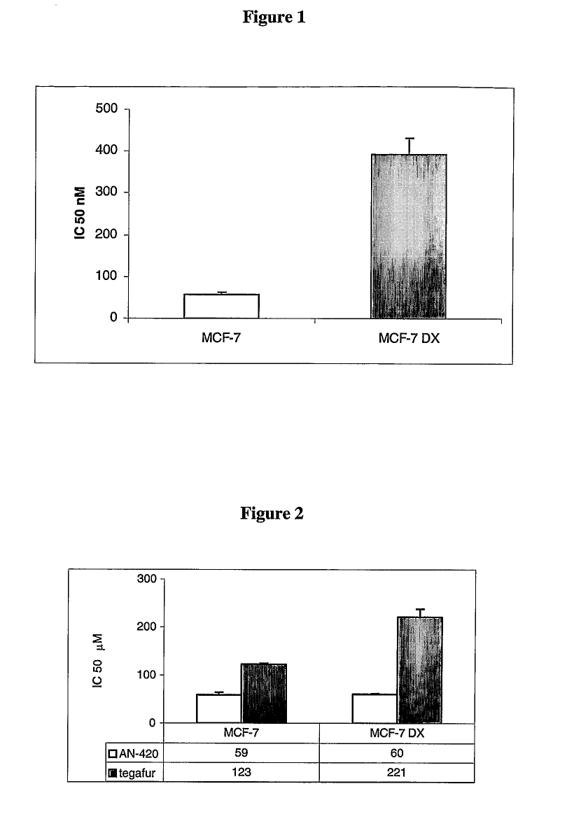

Resistant nature of cell lines: The resistant nature of various cancer cells was demonstrated and is presented in FIGS. 1-7.

Thus, for example, as is shown in FIG. 1, comparable IC₅₀ values of the anti-cancer chemotherapeutic agent Doxorubicin (Dox) with respect to the isogenetic breast carcinoma MCF-7 DX and MCF-7 cell lines show that the sub-clone MCF-7 DX, with which an average IC₅₀ value of 392±40 μM was observed in 7 independent experiments, is approximately 7 fold more resistant to doxorubicin, as compared with MCF cells, with which an IC₅₀ value of 58±6 μM was observed in 1 independent experiments. Similarly, as is shown in FIG. 2, in three independent experiments, the IC₅₀ of tegafur was significantly higher in the resistant MCF-7 DX cells.

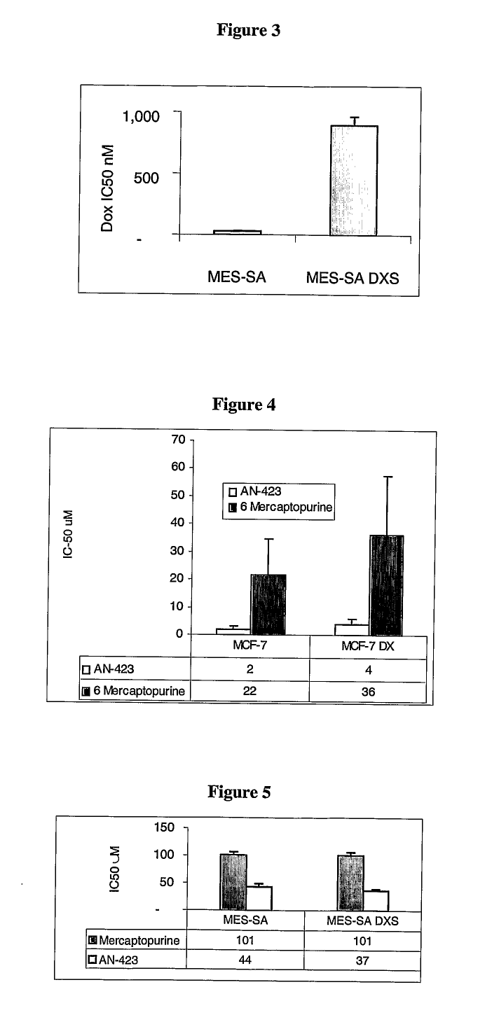

Drug Resistance is also most evident for the uterine carcinoma cells MES-SA DXS and MES-SA cell lines in response to the anticancer chemotherapeutic agent Doxorubicin (Dox). As is shown in FIG. 3, approximately 30 fold decrease in the inhibitory effect of doxorubicin on the proliferation of the resistant uterine sarcoma cells MES-SA DXS, as compared with the isogenetic non-resistant cells line, was observed.

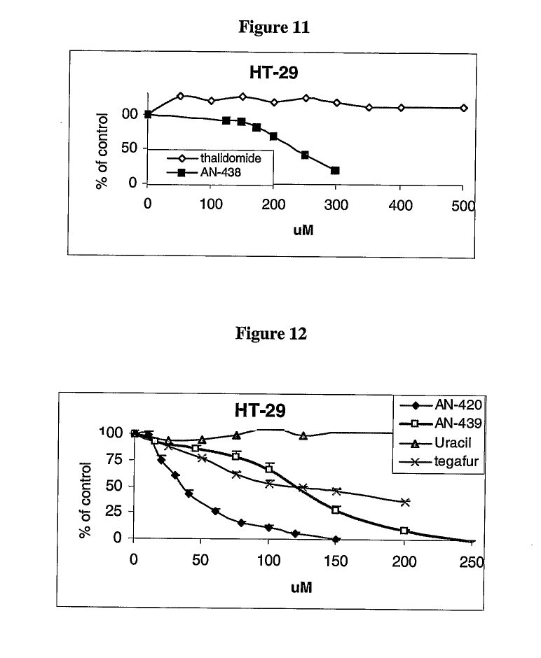

Anti-proliferative activity: The effect of the novel derivatives according to the present embodiments on sensitive and resistant cancer cells, in terms of IC₅₀ and/or IC₉₀ (measured by the proliferation assay described above) and/or the effect on the cells viability (measured by the apoptosis assay described above) is demonstrated in FIGS. 2-12.

Thus, FIG. 4 presents the effect of the 6-mercaptopurine derivative AN-423, according to the present embodiments, as compared with the effect of the unmodified 6-mercaptopurine, on the proliferation of human sensitive and resistant (MDR) breast cancer cells. As is shown in FIG. 4, the anti-proliferative activity of AN-423 was found to be approximately 10 fold greater than that of the parent compound in both sensitive and resistant cell lines.

FIG. 5 yet further presents the effect of the 6-mercaptopurine derivative according to the preferred embodiments of the present invention, AN-423, as compared with the effect of the unmodified 6-mercaptopurine, on the proliferation of human sensitive and resistant (MES) uterine cancer cells. As is shown in FIG. 5, the anti-proliferative activity of AN-423 was found to be approximately 2 fold greater than that of the parent compound in both sensitive and resistant cell lines.

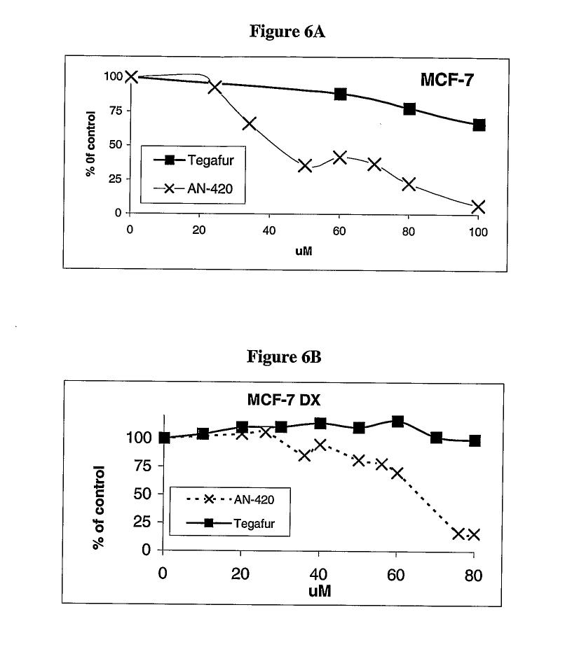

FIGS. 6A and 6B present the effect of the tegafur derivative according to the preferred embodiments of the present invention, AN-420, as compared with the effect of the unmodified tegafur, on the survival of both sensitive and resistant breast cancer. As is shown in FIGS. 6A and 6B, the tegafur derivative AN-420 affected the survival of both sensitive and resistant breast cancer cells to the same degree, but with a significantly lower IC₅₀ (p<<0.05, see, FIG. 2), demonstrating that AN-420 is appreciably more potent than the parent compound tegafur and overcomes drug resistance.

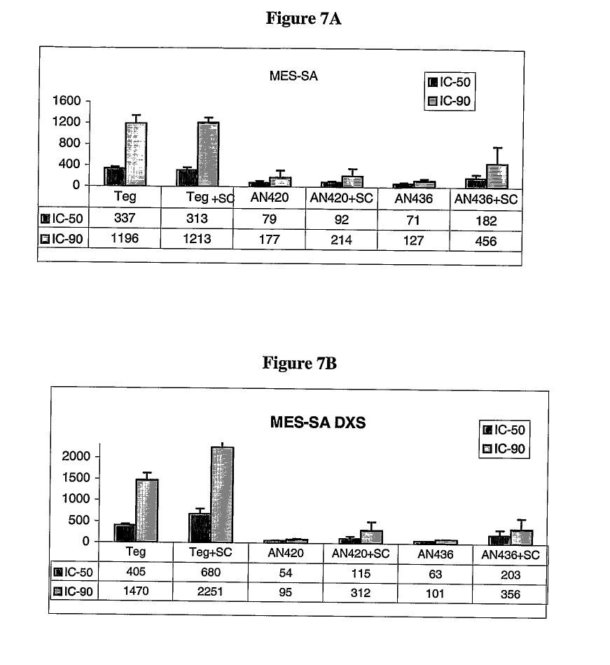

The effect of the tegafur derivatives according to the preferred embodiments of the present invention, AN-420 and AN-436, compared with the effect of the unmodified tegafur, was further tested on the survival of both sensitive and resistant uterine cancer cells. As is shown in FIGS. 7A and 7B, the tegafur derivatives AN-420 and AN-436 were 5-14 fold more potent than the parent compound tegafur to which the multidrug resistant uterine sarcoma cell line has a lower sensitivity. Moreover, while the tegafur derivatives accomplished 100% cell killing, the IC₉₀ values for tegafur were calculated by extrapolation from the formula of the best fitted curve. Experimentally tegafur achieved a maximum of 80% killing. The latter has an important implication in cancer therapy meaning that residual cancer cell population could survive the highest achievable dose of tegafur, while significantly lower dose of the derivatives can cause 100% mortality.

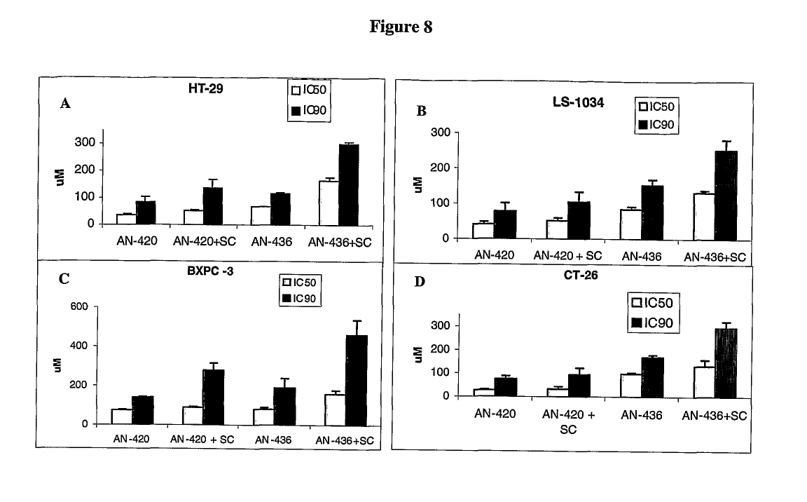

A similar effect is presented in FIG. 8: the effect of the tegafur derivatives AN-420 and AN-436 was further examined, as compared with the effect of the unmodified tegafur, on the survival of both sensitive and resistant colon cancer cells (LS-1034 and HT-29) and pancreatic cell line (BXPC3 and CT-26). As is shown in FIGS. 8A and 8B, the tegafur derivatives AN-420 and AN-436 were at least 5 and 2.5 fold, respectively (based on IC₅₀ values), and 10 and 5 fold, respectively, (based on IC₉₀ values) more potent than the parent compound tegafur. Furthermore, these finding demonstrate the greater potency of the tegafur derivative AN-420 compared with AN-436, against colon cancer cells.

In the human pancreatic cell line, BXPC3 comparable results were obtained, however the IC₅₀s of AN-420 and AN-436 did not differ significantly, while the IC₉₀ results did differ (FIGS. 8C and 8D).

As is discussed in detail hereinabove, the conjugates of the present embodiments are designed so as to release formaldehyde upon cleavage. It is thus postulated that these compounds are decomposed in situ into the chemotherapeutic agent, formaldehyde, and optionally another moiety that may exhibit a biological beneficial effect. For example, the tegafur derivative AN-420 decomposes to yield tegafur, formaldehyde and butyric acid, and the tegafur derivative AN-436 is decomposed to yield tegafur and formaldehyde.

As has already been shown (see, for example, Nudelman et al., J. Med. Chem., 2005, 48, 1047-1054), the presence of formaldehyde and e.g., butyric acid contribute to the activity of the chemotherapeutic agent. For example, the butyric acid may act by inhibiting histone deacetylase (HDAC) activity which leads to induced cellular death and differentiation.

Semicarbazide sensitive amine oxidase is a source of formaldehyde and is therefore a marker for the effect of formaldehyde. It has been shown that semicarbazide traps formaldehyde and reduces its concentration inside the cell (Nudelman et al. J. Med. Chem., 2005, 48, 1047-1054). Thus, the critical role of formaldehyde can be confirmed by reversal of formaldehyde-mediated effects by the addition of semicarbazide.

Tables 3 and 4 below, as well as FIGS. 7A, 7B and 8A-8D present the dose response studies performed with various cell lines in the absence and presence of semicarbazide, the formaldehyde trapping agent.

Co-treatment with semicarbazide (0.5 mM) substantially reduced the cell killing by AN-420 and reduced to an even greater extent of the cell killing by AN-436 (up to 3.5 folds). Semicarbazide alone, at 0.5 mM did not affect the cells, yet it reduced the IC₅₀ and IC₉₀ of AN-436 and to a significantly lesser extent of AN-420. Treatment with semicarbazide, in the absence of the chemotherapeutic agents, did not alter the cell viability. Therefore, it can be concluded that formaldehyde, released in situ, plays an important role in the reduction of cancer cell viability.

TABLE 3
The IC₅₀ values (μM) of Tegafur and its derivatives in various cell lines

| | | | AN-420 + SC | | AN-436 + SC |
|---|---|---|---|---|---|
| Cell line | Tegafur | AN-420 | 500 μM | AN-436 | 500 μM |
| HT-29 | 201 ± 15 | 35 ± 5 | 51 ± 5 | 67 ± 2 | 164 ± 12 |
| LS-1034 | 519 ± 93 | 42 ± 7 | 52 ± 8 | 84 ± 8 | 132 ± 7 |
| CT-26 | 136 ± 19 | 27 ± 4 | 34 ± 10 | 99 ± 5 | 132 ± 26 |
| BXPC-3 | 172 ± 21 | 74 ± 4 | 88 ± 6 | 81 ± 9 | 160 ± 17 |
| Panc 02 | 179 ± 25 | 46 ± 3 | | 96 ± 9 | |
| MES SA | 337 ± 20 | 79 ± 22 | 92 ± 14 | 71 ± 12 | 182 ± 18 |
| MES SA DXS | 405 ± 20 | 54 ± 5 | 115 ± 29 | 63 ± 4 | 203 ± 75 |

TABLE 4
The IC90 values (μM) of Tegafur and its derivatives in various cell lines

| | | | AN-420 + SC | | AN-436 + SC |
|---|---|---|---|---|---|
| Cell line | Tegafur | AN-420 | 500 μM | AN-436 | 500 μM |
| HT-29 | 747 ± 52 | 84 ± 21 | 138 ± 30 | 115 ± 7 | 303 ± 7 |
| LS-1034 | 1602 ± 240 | 79 ± 23 | 105 ± 29 | 153 ± 16 | 252 ± 30 |
| CT-26 | 411 ± 23 | 76 ± 14 | 94 ± 30 | 170 ± 9 | 296 ± 28 |
| BXPC-3 | 688 ± 113 | 141 ± 5 | 280 ± 36 | 191 ± 47 | 460 ± 78 |
| Panc 02 | 637 ± 66 | 149 ± 19 | | 219 ± 25 | |
| MES SA | 1196 ± 97 | 177 ± 77 | 214 ± 80 | 127 ± 19 | 456 ± 189 |
| MES SA DXS | 1470 ± 96 | 95 ± 8 | 312 ± 116 | 101 ± 2 | 356 ± 144 |

The obtained results obtained in these studies indicate the following: The conjugates of the present invention are significantly more potent than tegafur, as is particularly reflected in the observed IC₉₀ values; and

In most of the tested cell lines (HL-29, CT-26, Panc02 and LS1034), AN-420 is significantly more potent than AN-436, indicating a role for the butyric acid that is released in addition to the formaldehyde and/or a more efficient formaldehyde-releasing mechanism in case of such a derivative.

It is likely that in these cells, the butyric acid released from AN-420, contribute to the activity of the chemotherapeutic agent by inhibiting histone deacetylase (HDAC) activity, leading to induced cellular death and differentiation. Indeed, increase of histone acetylation in cells treated with AN-420, was demonstrated.

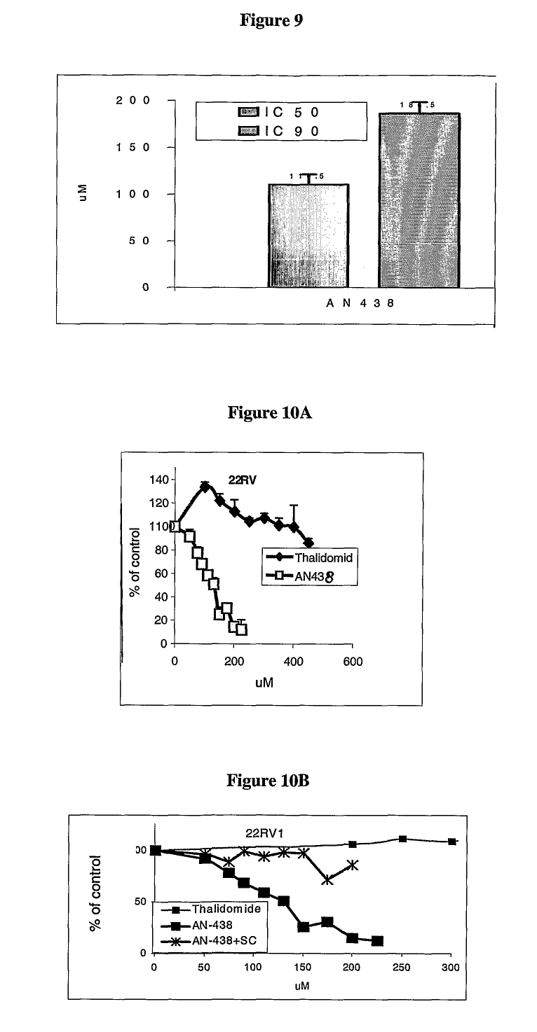

FIGS. 9-11 demonstrate that AN-438, a derivative of thalidomide, is significantly more effective in reducing the viability of the human prostate and colon carcinoma cell lines. Moreover, in the prostate carcinoma cells it is shown that the addition of 0.5 mM semicarbazide abrogated the effect of AN-438, demonstrating the central role of the released formaldehyde in the anticancer activity.

FIG. 12 presents the effect of the tegafur derivative AN-420, and of the uracil derivative AN-439 on the viability (as % of control) of colon carcinoma cells. As is shown In FIG. 12, uracil had no effect on the cancer cell viability, while its derivative, AN-439, reduced cell viability in a dose dependent manner. The butyroyloxymethyl derivative of tegafur was significantly more effective than tegafur in reducing the viability of these cells. Taken together it is evident that butyroyloxymethylation of pyrimidine-based chemotherapeutic agents such as uracil, tegafor and thalidomide increases their anticancer activity significantly.

In Vivo Studies:

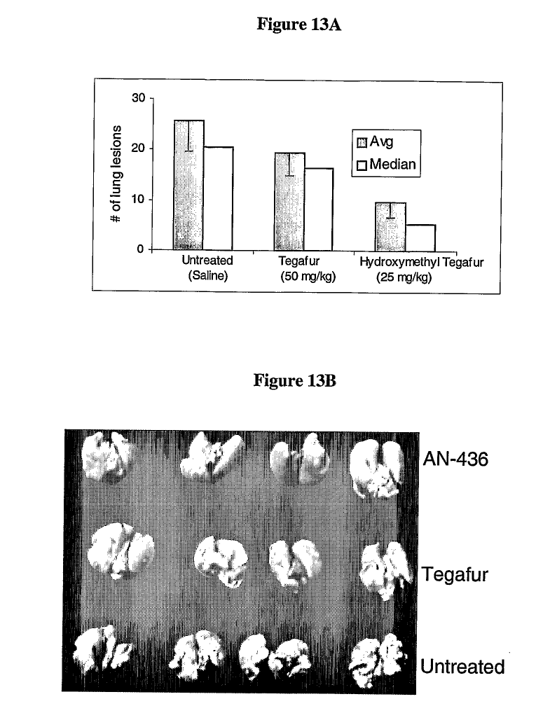

The in vivo anticancer efficacy of the tegafur derivative AN-436, according to the present embodiments, was compared with the effect of the unmodified tegafur, in the Lewis lung carcinoma model. As is shown in FIGS. 13A and 13B, the antimetastatic activity of AN-436 was significantly evident only in the animals treated with the tegafur derivative AN-436, and not in the animals treated with the parent compound.

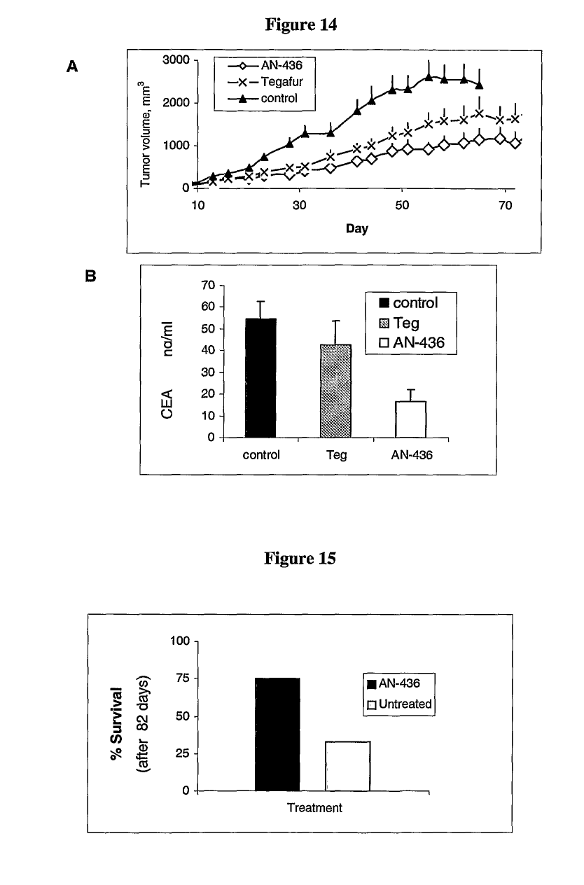

HT-29 is an accepted model for human colonic adenocarcinoma. The in vivo anticancer efficacy of the tegafur derivative AN-436, was demonstrated using the HT-29 model, compared with the effect of the unmodified tegafur. As is shown in FIG. 14A, although both the parent compound and AN-436 substantially inhibited tumor growth (P<<0.001), it was shown that the inhibitory effect of AN-436 was significantly higher than the parent compound tegafur with p<0.05.

The effect of the tegafur derivative AN-436 was further demonstrated using carcinoembryonic antigen (CEA), a common tumor marker. As is shown in FIG. 14B, the concentration of CEA was higher in the serum of untreated and mice treated with the parent compound tegafur-, compared with its level in the serum of mice treated with the tegafur derivative AN-436. Only treatment with AN-436 reduced the level significantly. Since the level of CEA in the serum correlates with tumor burden, it further indicates that AN-436 has greater anti-cancer activity in this in vivo cancer model.

As is shown in FIG. 15, studies on the effect of AN-436 on nude mice subcutaneously (sc) implanted with human colon carcinoma cells (LS1034) showed that after 82 days 75% of the treated animals survived and only 33% of the untreated mice survived. These results show that AN-436 also increased the survival of the animals in the resistant human carcinoma xenograft model (FIG. 15).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A conjugate comprising a first moiety and a second moiety, wherein said first moiety is a chemotherapeutic agent residue and said second moiety is selected such that the conjugate is capable of releasing at least one formaldehyde molecule and/or a formaldehyde analog molecule upon cleavage,
with the proviso that neither of said first moiety and said second moiety comprises a psychotropic drug residue, and further with the proviso that when said chemotherapeutic agent is 5-fluorouracyl, said second moiety is not a hydroxyalkyl, acyloxyalkyl or an alkoxycarbonyloxyalkyl.

2. The conjugate of claim 1, wherein said second moiety is selected such that said conjugate is further capable of releasing a biologically active moiety upon cleavage.

3. The conjugate of claim 2, wherein said biologically active moiety is selected from the group consisting of a carrier moiety, a therapeutically active moiety, a recognition moiety, and a formaldehyde-releasing group.

4. The conjugate of claim 1, wherein said second chemical moiety is selected from the group consisting of a hydroxyalkyl residue, a carboxyalkyl residue, and a first carboxyalkyl residue being covalently linked to a second carboxyalkyl residue.

5. The conjugate of claim 4, wherein said first carboxyalkyl residue is covalently linked to said second carboxyalkyl residue via a spacer.

6. The conjugate of claim 4, wherein each of said first and second carboxyalkyl residue is capable of releasing formaldehyde.

7. The conjugate of claim 1, wherein said chemotherapeutic agent is selected from the group consisting of an anti-cancer agent, an anti-viral agent, an immunomodulating agent, an anti-microbial agent, an anti-mycotic agent, an anti-helminthic agent and an anti-protozal agent.

8. The conjugate of claim 1, wherein said chemotherapeutic agent has at least one functional group that is capable of forming a covalent bond with said second moiety.

9. The conjugate of claim 6, wherein said functional group is selected from the group consisting of hydroxy, amine and thiol.

10. The conjugate of claim 9, wherein said chemotherapeutic agent is selected from the group consisting of acyclovir, abacavir, carmustine, dacarbazine, didanosine, edoxudine, emtricitabine, floxuridine, fludarabine, gangciclovir, gemcitabine, idoxuridine, lamivudin, lomustine, MADU, Nevirapine, Penciclovir, procarbazine, Sorivudine, Stavudine, Tegafur, trifluridine, valaciclovir, zalcitabine, zidovudine, Capecitabine (Xeloda), Thalidomide, Hydroxyurea, Mitomycin C, Vinca, temozolomide, 5-fluorouracil, capecitabine, a nitroso urea, cyclophosphamide, linezolide, penicillin, cephalosporin, sulfa, sulfamethoxazole, fluorocytosine, tolnaftate, caspofungin, thioguanine and 3-mercaptopurine.

11. The conjugate of claim 1, having the general Formula: 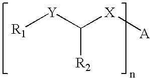
wherein:
n is an integer from 1 to 6;
XnA is a chemotherapeutic agent residue, whereas X is a residue of a functional group that forms a part of said chemotherapeutic agent;
R₂ is selected from the group consisting of hydrogen, alkyl, cycloalkyl and aryl;
Y is O or S; and
R₁ is selected from the group consisting of hydrogen, alkyl, cycloalkyl, aryl and a R₃C(═Z)- group, a (R₃G)₂P(═Z)- group and a R₃S(═Z)₂- group, whereas:
each of Z and G is independently O or S; and
R₃ is selected from the group consisting of hydrogen, alkoxy, arylalkyloxy, alkyl, cycloalkyl, aryl, aminoalkyl, haloalkyl, amine, alkylamine, dialkylamine, carboxyalkyl, carboxyethylene glycol, carboxyether, carboxythioether, ether and thioether.

12. The conjugate of claim 11, wherein n is an integer from 1 to 4.

13. The conjugate of claim 11, wherein n is an integer from 1 to 2.

14. The conjugate of claim 11, wherein X is selected from the group consisting of O, S and NR′, whereas R′ is hydrogen, alkyl, cycloalkyl and aryl.

15. The conjugate of claim 11, wherein R₂ is hydrogen.

16. The conjugate of claim 11, wherein Y is O.

17. The conjugate of claim 11, wherein each of Z and G is O.

18. The conjugate of claim 17, wherein R₁ is R₃C(═O)—.

19. The conjugate of claim 18, wherein R₃ is alkyl.

20. The conjugate of claim 19, wherein said alkyl is selected from the group consisting of methyl, ethyl, propyl, butyl, 2-methylpropyl, and tert-butyl.

21. The conjugate of claim 18, wherein R₃ is a dicarboxy being conjugated to carboxymethyl.

22. The conjugate of claim 18, wherein R₃ is aminoalkyl.

23. The conjugate of claim 22, wherein R₃ is aminopropyl.

24. The conjugate of claim 1, being selected from the group consisting of:
Butyric acid 5-fluoro-2,4-dioxo-3,4-dihydro-2H-pyrimidin-1-ylmethyl ester;
Butyric acid 5-fluoro-2,6-dioxo-3-(tetrahydro-furan-2-yl)-3,6-dihydro-2H-pyrimidin-1-ylmethyl ester;
Butyric acid 9H-purin-6-ylsulfanylmethyl ester;
Butyric acid 9-butyryloxymethyl-9H-purin-6-ylsulfanylmethyl ester;
Butyric acid 9-butyryloxy-9H-purin-6-ylsulfanylmethyl ester;
Butyric acid 2-(butyryloxymethyl-amino)-9H-purin-6-ylsulfanylmethyl ester;
Butyric acid-9-butyryloxymethyl-2-(butyryloxymethyl-amino)-9H-purin-6-ylsulfanylmethyl ester;
Pentanedioic acid butyryloxymethyl ester 5-fluoro-2,4-dioxo-3,4-dihydro-2H-pyrimidin-1-ylmethyl ester;
Butyric acid (5-fluoro-2,4-dioxo-3,4-dihydro-2H-pyrimidin-1-ylmethoxycarbonylmethoxy)-acetoxymethyl ester;
Butyric acid {2-[2-(5-fluoro-2,4-dioxo-3,4-dihydro-2H-pyrimidin-1-ylmethoxycarbonylmethoxy)-ethoxy]-ethoxy}-acetoxymethyl ester;
Pentanedioic acid butyryloxymethyl ester 5-fluoro-2,6-dioxo-3-(tetrahydro-furan-2-yl)-3,6-dihydro-2H-pyrimidin-1-ylmethyl ester;
Butyric acid [5-fluoro-2,6-dioxo-3-(tetrahydro-furan-2-yl)-3,6-dihydro-2H-pyrimidin-1-ylmethoxycarbonylmethoxy]-acetoxymethyl ester;
Butyric acid [5-fluoro-2,6-dioxo-3-(tetrahydro-furan-2-yl)-3,6-dihydro-2H-pyrimidin-1-ylmethoxycarbonylmethoxy]-acetoxymethyl ester;
5-Fluoro-3-hydroxymethyl-1-(tetrahydro-furan-2-yl)-1 H-pyrimidine-2,4-dione;
Diethyl (5-fluoro-2,3-dihydro-3-(tetrahydrofuran-2-yl)-2,6-dioxopyrimidin-1(6H)-yl)methyl phosphate;
2-(1-Butyroyloxymethyl-2,6-dioxopiperidin-3-yl)isoindoline-1,3-dione;
(5-fluoro-2,3-dihydro-3-(tetrahydrofuran-2-yl)-2,6-dioxopyrimidin-1(6H)-yl)methyl 4-aminobutanoate;
2-(1-(Hydroxymethyl)-2,6-dioxopiperidin-3-yl)isoindoline-1,3-dione;
2-(1-(Hydroxymethyl)-2,6-dioxopiperidin-3-yl)isoindoline-1,3-dione 4-aminobutanoate; and
Diethyl 2-(1-(hydroxymethyl)-2,6-dioxopiperidin-3-yl)isoindoline-1,3-dione phosphate.

25. A pharmaceutical composition comprising the conjugate of claim 1 and a pharmaceutically acceptable carrier.

26. The pharmaceutical composition of claim 25, being packaged in packaging material and identified in print, in or on said packaging material, for use in the treatment of a medical condition selected from the group consisting of proliferative disorder or disease, cancer, an immune-mediated disease, a viral infection or disease, a bacterial infection or disease, a fungal infection or disease, a protozal infection or disease, and a helminthic infection or disease.

27. The pharmaceutical composition of claim 26, wherein said condition is associated with drug resistance.

28. A method of treating a medical condition selected from the group consisting of proliferative disorder or disease, cancer, an immune-mediated disease, a viral infection or disease, a bacterial infection or disease, a fungal infection or disease, a protozal infection or disease, and a helminthic infection or disease, the method comprising administering to a subject in need thereof a therapeutically effective amount of a conjugate of a first moiety and a second moiety being covalently attached therebetween, wherein said first moiety is a chemotherapeutic agent residue and said second moiety is selected such that said conjugate is capable of releasing at least one formaldehyde molecule and/or a formaldehyde analog molecule upon cleavage,
with the proviso that neither of said first moiety and said second moiety comprises a psychotropic drug residue.

29-30. (canceled)

31. The method of claim 28, wherein said condition is associated with drug resistance.

32. The method of claim 28, wherein said second moiety is selected such that said conjugate is further capable of releasing a biologically active moiety upon cleavage.

33. The method of claim 32, wherein said biologically active moiety is selected from the group consisting of a carrier moiety, a therapeutically active moiety, a recognition moiety, and a formaldehyde-releasing group.

34. The method of claim 28, wherein said second chemical moiety is selected from the group consisting of a hydroxyalkyl residue, a carboxyalkyl residue, and a first carboxyalkyl residue being covalently linked to a second carboxyalkyl residue.

35. The method of claim 34, wherein said first carboxyalkyl residue is covalently linked to said second carboxyalkyl residue via a spacer.

36. The method of claim 34, wherein each of said first and second carboxyalkyl residue is capable of releasing formaldehyde.

37. The method of claim 28, wherein said chemotherapeutic agent is selected from the group consisting of an anti-cancer agent, an anti-viral agent, an anti-microbial agent, an anti-mycotic agent, an anti-helminthic agent and an anti-protozal agent.

38. The method of claim 28, wherein said chemotherapeutic agent has at least one functional group that is capable of forming a covalent bond with said second moiety.

39. The method of claim 38, wherein said functional group is selected from the group consisting of hydroxy, amine and thiol.

40. The method of claim 39, wherein said chemotherapeutic agent is selected from the group consisting of acyclovir, abacavir, carmustine, dacarbazine, didanosine, edoxudine, emtricitabine, floxuridine, fludarabine, gangciclovir, gemcitabine, idoxuridine, lamivudin, lomustine, MADU, Nevirapine, Penciclovir, procarbazine, Sorivudine, Stavudine, Tegafur, trifluridine, valaciclovir, zalcitabine, zidovudine, Capecitabine (Xeloda), Thalidomide, Hydroxyurea, Mitomycin C, Vinca, temozolomide, 5-fluorouracil, capecitabine, a nitroso urea, cyclophosphamide, linezolide, penicillin, cephalosporin, sulfa, sulfamethoxazole, fluorocytosine, tolnaftate, caspofungin, thioguanine and 3-mercaptopurine.

41. The method of claim 28, wherein said conjugate has the general Formula: 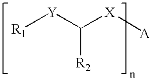
wherein:
n is an integer from 1 to 6;
XnA is a chemotherapeutic agent residue, whereas X is a residue of a functional group that forms a part of said chemotherapeutic agent;
R₂ is selected from the group consisting of hydrogen, alkyl, cycloalkyl and aryl;
Y is O or S; and
R₁ is selected from the group consisting of hydrogen, alkyl, cycloalkyl, aryl and a R₃C(═Z)- group, a (R₃G)₂P(═Z)- group and a R₃S(═Z)₂- group, whereas:
each of Z and G is independently O or S; and
R₃ is selected from the group consisting of hydrogen, alkoxy, arylalkyloxy, alkyl, cycloalkyl, aryl, aminoalkyl, haloalkyl, amine, alkylamine, dialkylamine, carboxyalkyl, carboxyethylene glycol, carboxyether, carboxythioether, ether and thioether.

42. The method of claim 41, wherein n is an integer from 1 to 4.

43. The method of claim 41, wherein n is an integer from 1 to 2.

44. The method of claim 41, wherein X is selected from the group consisting of O, S and NR′, whereas NR′ is hydrogen, alkyl, cycloalkyl and aryl.

45. The method of claim 41, wherein R₂ is hydrogen.

46. The method of claim 41, wherein Y is O.

47. The method of claim 41, wherein each of Z and G is O.

48. The method of claim 47, wherein R₁ is R₃C(═O)—.

49. The method of claim 48, wherein R₃ is alkyl.

50. The method of claim 49, wherein said alkyl is selected from the group consisting of methyl, ethyl, propyl, butyl, 2-methylpropyl, and tert-butyl.

51. The method of claim 48, wherein R₃ is a dicarboxy being conjugated to carboxymethyl.

52. The method of claim 48, wherein R₃ is aminoalkyl.

53. The method of claim 52, wherein R₃ is aminopropyl.

54. The method of claim 28, wherein said conjugate is selected from the group consisting of:
Butyric acid 5-fluoro-2,4-dioxo-3,4-dihydro-2H-pyrimidin-1-ylmethyl ester;
Butyric acid 5-fluoro-2,6-dioxo-3-(tetrahydro-furan-2-yl)-3,6-dihydro-2H-pyrimidin-1-ylmethyl ester;
Butyric acid 9H-purin-6-ylsulfanylmethyl ester;
Butyric acid 9-butyryloxymethyl-9H-purin-6-ylsulfanylmethyl ester;
Butyric acid 9-butyryloxy-9H-purin-6-ylsulfanylmethyl ester;
Butyric acid 2-(butyryloxymethyl-amino)-9H-purin-6-ylsulfanylmethyl ester;
Butyric acid-9-butyryloxymethyl-2-(butyryloxymethyl-amino)-9H-purin-6-ylsulfanylmethyl ester;
Pentanedioic acid butyryloxymethyl ester 5-fluoro-2,4-dioxo-3,4-dihydro-2H-pyrimidin-1-ylmethyl ester;
Butyric acid (5-fluoro-2,4-dioxo-3,4-dihydro-2H-pyrimidin-1-ylmethoxycarbonylmethoxy)-acetoxymethyl ester;
Butyric acid {2-[2-(5-fluoro-2,4-dioxo-3,4-dihydro-2H-pyrimidin-1-ylmethoxycarbonylmethoxy)-ethoxy]-ethoxy}-acetoxymethyl ester;
Pentanedioic acid butyryloxymethyl ester 5-fluoro-2,6-dioxo-3-(tetrahydro-furan-2-yl)-3,6-dihydro-2H-pyrimidin-1-ylmethyl ester;
Butyric acid [5-fluoro-2,6-dioxo-3-(tetrahydro-furan-2-yl)-3,6-dihydro-2H-pyrimidin-1-ylmethoxycarbonylmethoxy]-acetoxymethyl ester;
Butyric acid [5-fluoro-2,6-dioxo-3-(tetrahydro-furan-2-yl)-3,6-dihydro-2H-pyrimidin-1-ylmethoxycarbonylmethoxy]-acetoxymethyl ester;
5-Fluoro-3-hydroxymethyl-1-(tetrahydro-furan-2-yl)-1H-pyrimidine-2,4-dione;
Diethyl (5-fluoro-2,3-dihydro-3-(tetrahydrofuran-2-yl)-2,6-dioxopyrimidin-1(6H)-yl)methyl phosphate;
2-(1-Butyroyloxymethyl-2,6-dioxopiperidin-3-yl)isoindoline-1,3-dione;
(5-fluoro-2,3-dihydro-3-(tetrahydrofuran-2-yl)-2,6-dioxopyrimidin-1(6H)-yl)methyl 4-aminobutanoate;
2-(1-(Hydroxymethyl)-2,6-dioxopiperidin-3-yl)isoindoline-1,3-dione;
2-(1-(Hydroxymethyl)-2,6-dioxopiperidin-3-yl)isoindoline-1,3-dione4-aminobutanoate; and
Diethyl 2-(1-(hydroxymethyl)-2,6-dioxopiperidin-3-yl)isoindoline-1,3-dione phosphate.

55. The method of claim 28, further comprising administering to said subject a therapeutically effective amount of an additional agent capable of treating the condition.

56. A process of preparing the conjugate of claim 1, the process comprising:
providing said chemotherapeutic agent;
providing a reactive derivative of said second moiety; and
reacting said chemotherapeutic agent and said reactive derivative, thereby providing the conjugate.

57. The process of claim 56, wherein said reacting is performed in the presence of a base.

58. The process of claim 56, further comprising, prior to said reacting:
providing a reactive derivative of said chemotherapeutic agent.

59. The process of claim 58, wherein said second moiety and is not a hydroxyalkyl moiety and said reactive derivative of said chemotherapeutic agent is a hydroxyalkyl derivative of said chemotherapeutic agent.
